(19) United States
(12) Patent Application Publication
(10) Pub. No.: US 2007/0224128 A1
(43) Pub. Date: Sep. 27, 2007

(54) Drug adherence monitoring system

(76) Inventors: Donn Michael Dennis, Gainesville, FL (US); Richard J. Melker, Gainesville, FL (US); Matthew M. Booth, Gainesville, FL (US); Laszlo Prokai, Mansfield, TX (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/715,197

(22) Filed: Mar. 7, 2007

Related U.S. Application Data
(60) Provisional application No. 60/779,729, filed on Mar. 7, 2006.

(51) Int. Cl. A61K 49/00; G01N 33/497

(52) U.S. Cl. 424/10.1; 73/23.3

(57) ABSTRACT

The present invention provides novel methods for monitoring subject adherence in taking prescribed drugs by detecting markers in exhaled breath after a subject takes the prescribed drug. In particular, the present invention provides novel methods for making additives that are combined with the drug(s). Upon biological breakdown of the drug/additive formulation in a subject's body, markers resulting directly from the biological breakdown of the additives are detected in exhaled breath using sensor technology. In certain embodiments of the invention, the drug adherence monitoring systems and methods include a reporting system capable of tracking subject compliance (either remotely or proximately) and of providing necessary alerts to the subject, caregiver, healthcare provider, and the like.

### CROSS-REFERENCE TO A RELATED APPLICATION

This application claims the benefit of U.S. provisional application Ser. No. 60/779,729, filed Mar. 7, 2006, which is hereby incorporated by reference in its entirety.

### FIELD OF INVENTION

The present invention relates to marker detection, in the form of odors or the like, to monitor drug adherence, and, more particularly, to a method and apparatus for the detection of markers in exhaled breath after the drug is taken by a subject, wherein such markers are combined with the drug.

### BACKGROUND INFORMATION

Breath is a unique bodily fluid. Unlike blood, urine, feces, saliva, sweat and other bodily fluids, it is available on a breath to breath and therefore continuous basis. It is readily available for sampling non-invasively and because the lung receives all of the blood flow from the right side of the heart, it has been suggested that measurements of analytes/compounds in breath correlate with blood concentration. Another positive aspect of breath sampling, as opposed to other bodily fluids, is that breath is less likely to be associated with the transfer of serious infections. Further, the collection of breath samples is relatively straightforward and painless.

Further, exhaled breath contains 100% humidity at 37° C. (body temperature), thus it can be considered an aerosol. If the temperature of the collected sample is maintained at 37° C. or higher it will remain in this state and can be treated as a gas for compounds that are insoluble in water or readily diffuse out of water. In this instance, sensors designed to work with gaseous media would be preferable. For compounds that are highly water soluble and likely to remain in solution, the exhaled breath sample can be collected as a condensate when cooled. This liquid can then be analyzed with sensors that are designed for liquid-based analyses. Compounds likely to be detectable in the gas phase typically are lipophilic (hydrophobic) such as the intravenous anesthetic agent, propofol, while compounds likely to be detected in the liquid phase are hydrophilic, such as glucose, lactic acid and perhaps even electrolytes. Thus an exhaled breath sample can be handled to produce a gaseous matrix for certain compounds and sensors, and a liquid matrix for others. In instances where it is desirable to detect more than one compound (e.g., detection of hydrophilic and hydrophobic molecules in the breath), the sample can be split and a portion maintained as a gas and a portion condensed as a liquid.

It is well-established in the medical literature that the actions of prescription drugs depend upon the amount (dose) of drug taken and the time intervals that separate successive doses of the drug. Drug non-compliance (or non-adherence) is the failure to take drugs on time in the dosages prescribed, which results in subject underdrug or overdrug. Lack of drug adherence is as dangerous and costly as many illnesses. As any physician or caregiver understands, medicine is only effective when taken as prescribed.

Noncompliance cuts across all categories of subjects and illnesses. People with breast cancer, organ transplants, and hypertension, as well as people on a short course of antibiotics, can all forget to take their drugs. Researchers have identified more than 200 variables that affect whether a subject will be compliant. Compliance rates are also likely to decline over time, especially for subjects with asymptomatic diseases.

Non-compliance of subjects to drug regimens prescribed by their physicians results in excessive healthcare costs estimated to be around $100 billion per year through lost work days, increased cost of medical care, higher complication rates, as well as drug wastage. Studies have shown that non-compliance causes 125,000 deaths annually in the U.S. alone [Smith, D., “Compliance Packaging: A Subject Education Tool,” American Pharmacy, NS29(2) (1989)]. Moreover, drug non-adherence leads to 10 to 25 percent of hospital and nursing home admissions, and is becoming an international epidemic [Standberg, L. R., “Drugs as a Reason for Nursing Home Admissions,” American Healthcare Association Journal, 10(20) (1984); Schering Report IX, The Forgetful Subject: The High Cost of Improper Subject Compliance; Oregon Department of Human Resources, A study of Long-Term Care in Oregon with Emphasis on the Elderly, (March 1981)].

About 50% of the 2 billion prescriptions filled each year are not taken correctly [National Council for Subject Information and Education]. ⅓ of subjects take all their medicine, ⅓ or subjects take some dosage of the prescribed medicine, ⅓ of subjects do not take any at all [Hayes, R. B., NCPIE Prescription Month, (October 1989)]. Such sub-optimal rates of compliance reported by various studies becomes of even greater concern as the American populace ages and becomes more dependent on drugs to fight the illnesses accompanying old age. By 2025, over 17% of the US population will be over 65 [Bell J A, May F E, Stewart R B: Clinical research in the elderly: Ethical and methodological considerations. Drug Intelligence and Clinical Pharmacy, 21: 1002-1007, 1987] and senior citizens take, on average, over three times as many drugs compared to the under 65 population [Cosgrove R: Understanding drug abuse in the elderly. Midwife, Health Visitor & Community Nursing 24(6):222-223, 1988]. The forgetfulness that sometimes accompanies old age also makes it even more urgent to devise cost-effective methods of monitoring compliance on a large scale.

Further, non-compliance of subjects with communicable diseases costs the public health authorities millions of dollars annually and increases the likelihood of drug-resistance, with the potential for widespread dissemination of drug-resistant pathogens resulting in epidemics. For example, one of the most serious consequences of noncompliance involves the outbreak of new, drug-resistant strains of HIV, which has been attributed to subjects who do not properly follow their complex drug regimens. In addition, the long-term misuse of antibiotics has given rise to forms of previously treatable diseases that are impervious to the most advanced drugs.

Current methods of improving drug adherence for health problems are mostly complex, labor-intensive, and not predictably effective [McDonald, H P et al., “Interventions to enhance subject adherence to drug prescriptions: scientific review,” JAMA, 289(4):3242 (2003)]. A cost-effective, but difficult to administer, program has been developed in seven locations around the nation to combat this serious threat to the American populace. It involves direct observation of all drug delivery by trained professionals (directly observed therapy: DOT) but is impractical for large scale implementation. Many techniques are also invasive, e.g., blood sampling.

Previous drug adherence monitoring systems disclosed by the present inventors related to the use of exhaled breath as a means to detect when and/or whether a subject has taken drug as prescribed (see, for example, U.S. patent application Ser. Nos. 10/722,620 (filed Nov. 26, 2003) and 11/097,647 (filed Apr. 1, 2005)). The monitoring systems described in those applications either detected in exhaled breath the drug; a metabolite of the drug; or a detectable marker (that was combined with the drug) or its metabolite. Many of the markers considered for use in those applications were largely GRAS (“Generally Recognized As Safe”) compounds, as classified by the FDA. Unfortunately, currently available detectors (sensors) do not detect these compounds in exhaled breath reliably and specifically in sufficient concentration to be used in practical devices.

Accordingly, there is a need in the art for a system and method to improve drug compliance which provides simple monitoring of drug dosing which is non-invasive, intuitive and sanitary. In particular, there is a need for a unique group of markers that can be combined with drug for adherence monitoring, where the markers are highly volatile and easily detectable in exhaled breath, even at very low concentrations using commercially available detectors.

### SUMMARY OF THE INVENTION

The present invention solves the needs in the art by providing a method and apparatus for non-invasive monitoring of drug adherence by detecting a marker in exhaled breath that is the product of drug absorption, distribution, metabolism, and/or excretion in the subject's body. Preferably, the markers are derived from an additive that is combined with the drug, wherein the markers are detectable in exhaled breath upon the absorption, distribution, metabolism, and/or excretion of both the drug and the additive by the subject.

According to the invention, markers can be detected in exhaled breath using any number of currently available sensor technologies. In one embodiment, the invention preferably utilizes commercial devices referred to as “artificial noses/electronic noses” or “electronic tongues,” to detect markers in exhaled breath and non-invasively monitor subject compliance in taking a drug.

In certain embodiments, the systems and methods of the invention not only detect markers, they also quantify and trend the concentration of the markers in exhaled breath that are indicative of subject compliance in taking a drug. According to one aspect of the invention, the concentration of the markers in exhaled breath can correlate with the concentration of drug taken by the subject, and thus enable non-invasive assessment of whether the appropriate drug dosage was taken by the subject.

The subject systems and methods of the invention include: at least one drug to be taken by a subject, wherein the drug includes an additive that when metabolized produces a marker detectable in exhaled breath; and an exhaled breath sensor for analyzing the subject's breath for the presence and/or concentration of at least one marker. The markers are indicative of the subject's compliance in taking the drug.

The methods of the subject invention include the steps of detecting and/or measuring the concentration of one or more markers in a subject's exhaled breath. The marker concentration in exhaled breath can be used to quantify the concentration (or dosage) of drug(s) in the subject's blood.

In certain embodiments of the subject invention, a specific phase of the respiratory cycle, namely the end-tidal portion of exhaled breath, is sampled to detect the presence and/or quantify the concentration of a marker as a measure of subject compliance in taking a drug. In other embodiments, liquid components found in exhaled breath are subjected to sensor technology to detect the presence and/or quantify the concentration of a marker.

Sensors used in accordance with the subject invention include, but are not limited to, commercial devices commonly known as “artificial” or “electronic” noses or tongues to non-invasively monitor drug adherence by a subject. Sensors of the subject invention can include, but are not limited to, metal-insulator-metal ensemble (MIME) sensors, cross-reactive optical microsensor arrays, fluorescent polymer films, corona devices, surface enhanced Raman spectroscopy (SERS), semiconductor gas sensor technology, conductive polymer gas sensor technology, surface acoustic wave gas sensor technology, functionalized microcantilevers, microspectrometers, and immunoassays.

The subject invention includes methods for the development of additives for combining with drugs, where additive by-products (also referred to herein as markers) resulting from subject bioactivity on the additives will appear in exhaled breath. The markers are used to determine in a foolproof manner whether a subject has ingested his/her drug as prescribed by their medical provider.

In certain embodiments, the systems of the subject invention include a reporting system capable of tracking marker presence/concentration (either remotely or proximately) and providing the necessary outputs, controls, and alerts. For example, in certain embodiments, the invention provides a reporting system capable of tracking subject compliance in taking one or more drugs (via marker detection in exhaled breath) and alerting the subject, healthcare personnel, and/or caregivers of non-compliance. Alerts to be provided can include an alarm and/or a report.

A drug adherence monitoring system of the subject invention can be used either in a clinical setting or subject-based location. Small handheld portable drug adherence monitoring system (MAMS) equipment could be used by subjects in the home, at work, in nursing homes, or while they are ambulatory, while other MAMS could be designed for continuous monitoring in the operating room, intensive care units and in other areas of hospitals or other healthcare facilities such as clinics, doctors offices where this capability would be valuable.

In one embodiment of the invention, monitoring of marker presence and/or concentration is conducted continuously using a system of the invention. In another embodiment of the invention, monitoring of marker presence and/or concentration is conducted intermittently using a system of the invention. In one example, a sensor of the subject invention would be used either in a healthcare setting or a remote subject-based location, to monitor appropriate delivery of drugs to a subject by detecting and/or measuring a target marker in subject exhaled breath.

Accordingly, the present invention provides a drug monitoring system that includes a computer that is programmed with a drug regimen of a particular subject and a sensor, wherein the computer has the capability to track and store sensor results, signal alarms, generate reports, and the like. At the time a drug is administered to a subject, a sample of the subject's exhaled breath is provided to the sensor, either via a voluntary exhaled breath or, if the subject is intubated through an endotracheal (ET) or tracheostomy tube, in which case the sensor is placed in line with the tube to detect and/or quantify the markers present in the subject's exhaled breath. With such a drug monitoring system, clinicians can record and track whether a subject has been properly medicated by a caregiver. Such a system could also prevent drug errors from occurring.

According to another aspect of the invention comprises a drug adherence monitoring kit for detecting the presence of target markers in exhaled breath, including: a housing; a sensor disposed within the housing, said sensor having the ability detect the presence of target markers and/or quantify marker concentration in exhaled breath; and a reporting module disposed within the housing adjacent to the sensor, wherein said reporting module is operatively connected to the sensor such that detection of the presence of the marker(s) and/or quantification of marker concentration in exhaled breath by the sensor is communicated to the user via the reporting module.

Therefore, it is an object of the present invention to non-invasively monitor subject compliance in taking drug(s) by monitoring the presence and/or concentration of a marker (associated with the drug) present in exhaled breath using sensors that analyze markers in exhaled breath.

A resulting advantage of the subject invention is the ability to monitor subject adherence in taking drugs in a non-invasive, easy-to-use, cost effective, and continuous manner. The subject invention specifically provides a system that better addresses the causes contributing to the inaccurate use of prescription drugs than those currently on the market. In addition, the subject invention enables decreased economic and societal costs associated with drug noncompliance, such as costs associated with decreased hospitalization due to increased drug efficacy and costs associated with addressing microbial resistance to drugs.

The invention will now be described, by way of example and not by way of limitation, with reference to the accompanying sheets of drawings and other objects, features and advantages of the invention will be apparent from the following detailed disclosure and from the appended claims.

### BRIEF DESCRIPTION OF THE DRAWINGS

FIG. 1 is an illustration of an additive ester group that is metabolized in the subject's body to an alcohol that is detectable in exhaled breath.

FIG. 2 is an illustration of another additive group that is metabolized via alkaline phosphatase in the subject's body to an alcohol that is detectable in exhaled breath.

FIG. 3 is a schematic illustration of the O-demethylation of dextromethorphan by CYP2D6.

FIG. 4 is a schematic illustration of the synthesis of an additive (O-trifluoroethyl dextrorphan) in accordance with one embodiment of the invention.

FIG. 5 is a graphical illustration of the inhibition of CYP 2D6 activities of AMMC due to increasing concentrations of dextromethorphan and trifluoroethyl dextrorphan from 10⁻¹⁰ M to 10⁻⁵ M.

FIG. 6 is a graphical illustration of the in vivo metabolism of an additive (O-trifluoroethyl dextrorphan) to yield a detectable, volatile marker compound (trifluoroacetaldehyde).

FIGS. 7A and B are graphical illustrations of total-ion chromatogram of trifluoroacetaldehyde 2,4-dinitrophenylhydrazone and its ¹⁵N₄-labeled internal standard, respectively, upon GC/MS analysis.

FIGS. 7C and D are graphical illustrations of full scan NCI mass spectra of trifluoroacetaldehyde 2,4-dinitrophenylhydrazone and its ¹⁵N₄-labeled internal standard, respectively, upon GC/MS analysis.

### DETAILED DESCRIPTION OF THE INVENTION

The present invention provides a method and apparatus for non-invasive monitoring of drug adherence by a subject by detecting a marker in exhaled breath that is the product of drug absorption, distribution, metabolism, and/or excretion in the subject's body. Preferably, the marker is detectable in exhaled breath after the drug is taken by the subject. In one embodiment, the detected markers are derived from a novel additive combined with the drug, where both the additive and the drug are absorbed, distributed, metabolized, and/or excreted in the subject's body.

Throughout this disclosure, the marker is defined as the by-product of a substance (also referred to herein as an additive) that is added to the drug to be taken by the subject as prescribed. Upon absorption, distribution, metabolism, and/or excretion of the substance/additive by the subject, the marker is detectable in exhaled breath. Preferably, the marker is detected in exhaled breath by means of its physical or chemical properties and is used as an indication that the subject has complied in taking the drug.

The present invention provides systems and methods for non-invasive monitoring of subject adherence in taking drug(s) by analyzing a subject's exhaled breath for the presence of a marker indicative of drug absorption, distribution, metabolism, and/or excretion in the subject's body.

In certain embodiments, the breath concentration of at least one marker is analyzed using sensor technology, wherein marker concentration correlates to the concentration of drug in the subject, particularly drug concentration in the blood (also referred to herein as drug monitoring [TDM]). Thus, based on the breath concentration of the markers, the concentration of the corresponding drug in a subject can be non-invasively and efficiently assessed. Knowledge of the drug concentration in the subject is particularly useful in assessing whether the appropriate drug dosage was taken by the subject.

### Definitions

As used herein, the term “drug” or “drug” refers to a substance used in the diagnosis, treatment, or prevention of a disease or condition, wherein the presence of the drug in the subject (or concentration of the drug in the subject's blood stream) is monitored to ensure subject compliance in taking the drug. A drug of the present invention includes drugs useful in the treatment of any one of the following conditions including, but not limited to, Attention Deficit Disorder (ADD or ADHD); adrenal disorders; AIDS and other viral illnesses; allergies; anxiety; bacterial infections; birth defects; blood disorders; cancer; cardiovascular disorders; depressive disorders; diabetes; digestive disorders; dyslexia; ear, nose and throat conditions; endocrine disorders; endometriosis; eye disorders; genetic disorders; genitourinary disorders; halitosis; hangover; hemorrhoids; hormonal disorders; immune disorders; infectious diseases; insulin resistance; musculoskeletal disorders; neurological disorders; nutrition disorders; parathyroid; parasitic infections; pituitary; polycystic ovarian syndrome; pregnancy complications; premature ejaculation; respiratory disorders; sexual transmitted diseases; skin disorders; sleep disorders; and thyroid.

Throughout this disclosure, a “marker” is defined as a substance that is detected in exhaled breath by means of its physical or chemical properties using a sensor of the subject invention. Markers of the invention are preferably unique in exhaled breath (for example, they are not molecules commonly present in exhaled breath, they are not found in foods, they are not endogenously generated, etc.); metabolically stable; non-toxic to the subject; do not alter the pharmacokinetics and/or pharmacodynamics of the drug; relatively inexpensive; readily available; and easy to synthesize as well as integrate with the drug.

Halogenated compounds (i.e. fluorinated markers) hold particular promise as they are readily highly volatile, safe for human consumption at doses required, and are readily detected in exhaled breath with several types of portable Freon leak detectors. Some of these compounds are used as propellants for delivery of drugs via the pulmonary route, such as metered dose inhalers and therefore are known to be safe and are FDA approved. The technologies most often used to detect Freon leaks include: Negative Ion Capture, Heated Sensor/Ceramic Semiconductor, Infrared Absorption, and TIF TIFXP-1A Negative Corona Leak Detector. Many drugs are fluorinated and metabolites are often extremely volatile and detectable in exhaled breath. Numerous such compounds are available that could be used as markers and could be added as excipients during the manufacture of drugs

A “subject,” as used herein, describes an organism, including mammals, from which exhaled breath samples are collected in accordance with the present invention. Mammalian species that benefit from the disclosed systems and methods for drug monitoring include, and are not limited to, apes, chimpanzees, orangutans, humans, monkeys; and domesticated animals (e.g., pets) such as dogs, cats, mice, rats, guinea pigs, and hamsters.

According to the subject invention, markers detectable in exhaled breath using the systems and methods of the invention include those that may be found in breath gas, breath condensate (liquid phase), respiratory droplet, breath evaporate, water vapor, and/or bronchial or alveolar aerosols.

The term “pharmacodynamics,” as used herein, refers to the interaction (biochemical and physiological) of a drug with constituents of a subject body as well as the mechanisms of drug action on the subject body (i.e., drug effect on body).

As used herein, the term “pharmacokinetics” refers to the mathematical characterization of interactions between normal physiological processes and a drug over time (i.e., body effect on drug). Certain physiological processes (absorption, distribution, metabolism, and elimination) will affect the ability of a drug to provide a desired effect in a subject. Knowledge of a drug's pharmacokinetics aids in interpreting drug blood stream concentration and is useful in determining pharmacologically effective drug dosages.

The term “aptamer,” as used herein, refers to a non-naturally occurring oligonucleotide chain that has a specific action on a drug marker. Aptamers include nucleic acids that are identified from a candidate mixture of nucleic acids. In a preferred embodiment, aptamers include nucleic acid sequences that are substantially homologous to the nucleic acid ligands isolated by the SELEX method. Substantially homologous is meant a degree of primary sequence homology in excess of 70%, most preferably in excess of 80%.

The “SELEX™” methodology, as used herein, involves the combination of selected nucleic acid ligands, which interact with a target marker in a desired action, for example binding to an olfactory marker, with amplification of those selected nucleic acids. Optional iterative cycling of the selection/amplification steps allows selection of one or a small number of nucleic acids, which interact most strongly with the target marker from a pool, which contains a very large number of nucleic acids. Cycling of the selection/amplification procedure is continued until a selected goal is achieved. The SELEX methodology is described in the following U.S. patents and patent applications: U.S. patent application Ser. No. 07/536,428 and U.S. Pat. Nos. 5,475,096 and 5,270,163.

As used herein, the term “pharmaceutically acceptable carrier” means a carrier that is useful in preparing a pharmaceutical composition that is generally compatible with the other ingredients of the composition, not deleterious to the subject, and neither biologically nor otherwise undesirable, and includes a carrier that is acceptable for veterinary use as well as human pharmaceutical use. “A pharmaceutically acceptable carrier” as used in the specification and claims includes both one and more than one such carrier.

### Drug Adherence Monitoring System

The subject invention relates to a system and method of drug adherence monitoring that includes regularly using a breath sensor for detecting drug markers in a sample of the subject's breath (for example, at prescribed intervals), where the drug marker is associated with a prescribed drug; and regularly (for example, at prescribed intervals in which exhaled breath samples are taken and applied to a sensor) assessing subject compliance with a prescribed drug regimen based on sensor results. Certain embodiments include intervening with the subject when appropriate to improve compliance. Appropriateness for intervention is dependent upon the detected concentration of markers in exhaled breath samples when compared against an expected marker concentration based on the prescribed drug regimen.

In certain embodiments, the marker can be indicative for the specific drug administered and/or for the specific prescribed dosage. For example, certain subjects may be prescribed various dosages of a particular type of drug. The MAMS of the invention can discern not only whether the subject has taken the drug, but also whether the subject has taken the correct dosage of a prescribed drug.

A drug adherence monitoring system (also referred to herein as MAMS) of the invention is guided by ongoing measurements of the subject's compliance with the prescribed drug regimen(s). An integral part of a MAMS involves the triggering of specific interventions, as derived from monitored levels of drug marker in exhaled breath, to improve subject compliance. A MAMS of the invention includes an apparatus for monitoring subject compliance with at least one drug regime, including a means for obtaining a sample of a subject's exhaled breath; a sensor for detecting at least one drug marker in the sample; and a means for processing detected drug marker(s), including a means for storing data regarding detected drug marker(s) and for assessing detected concentrations of drug marker(s) for monitoring and/or clinical applications (i.e., comparing detected concentration of drug marker(s) against expected concentration for the regime period, against previous recorded concentrations, and/or against other drug marker concentrations).

A method of using the MAMS of the invention includes sampling a subject's exhaled breath; applying a sensor to the exhaled breath sample to detect the presence of any drug markers; and assessing the detected concentration of drug markers in the sample against an expected concentration of drug markers for the prescribed drug.

Related methods for monitoring adherence can further include any one or combination of the following steps: analyzing data on the clinical consequences of variable subject compliance with the prescribed drug regimen(s); defining expected concentration of a drug marker in a sample based on prescribed drug period(s) or part thereof; altering, maintaining, canceling, or adding to the prescribed drug regimen for the subject; providing results regarding subject compliance to the user (which includes the subject, physician, or the like); assessing the subject's health status based on the pattern of drug compliance as provided by MAMS; assessing indicators of progression of subject condition while taking the prescribed regimen (such as assessing blood pressure; body weight or related indices of body size; plasma levels of cholesterol and its various fractions; parameters of diabetes control, including glycosylated hemoglobin levels or glucose concentrations in blood; and other biochemical and biophysical indicators); providing a drug that produces a marker detectable in exhaled breath; and intervening with the subject when appropriate to improve subject compliance.

In one embodiment, the MAMS of the invention are designed to function under the following medical and engineering constraints: 1) since the vast majority of drugs used in clinical medicine today are given orally once (PO QD) or twice (PO BID) per day, MAMS can be designed to function for either drug administration schedule; and 2) to provide the greatest benefit to subjects and to most rapidly bring MAMS technology to the broadest array of drug markets, a single MAMS can be constructed that functions for monitoring all orally administered drugs (versus a specific MAMS developed for each and every specific drug); and 3) a commercial-off-the-shelf (COTS) device or commercially available sensing technology can constitute the sensing component of the MAMS.

In related embodiments, the MAMS of the invention are portable; provide rapid (and in certain instances, real time), sensitive, and specific detection of markers and/or measurement of marker concentrations in the exhaled breath media; and/or can be coupled to existing well-developed technologies (e.g., biometrics, videophone) to ensure that ingestion of a drug occurred in a given subject. Accordingly, the measurement of marker concentration does not necessarily need to be quantitative; “semi-quantitative” measurements in certain embodiments are sufficient, so long as the measurements avoid overlap with previously MAMS assessed drug doses.

In other embodiments, MAMS are applied to drugs that are administered via non-oral modes of drug delivery (e.g., intravenous, ophthalmologic, dermatological, etc.).

According to the present invention, a sensor for use in detecting markers in exhaled breath can be operatively connected to a data processing system. The processing system is preferably programmed to assimilate and analyze output signals generated by the sensor regarding markers detected in exhaled breath samples. In one embodiment, the processing system is a computer. Marker analysis results can be displayed on a computer screen, stored, transmitted, etc. Moreover, a computer processing unit (or CPU) may be provided as a data processing/control unit. In one embodiment, the processing unit is programmed for conducting a comparison of data regarding recommended marker levels for a prescribed drug regimen against monitored drug marker data in subject exhaled breath samples to determine if there are any deviations from prescribed drug, dosage, and/or duration ranges for the drug.

In one embodiment, the CPU can automatically detect and store signals from the sensor to enable proper tracking and analysis of marker detection and/or marker concentration in exhaled breath. In a related embodiment, where a flow sensor is provided in operative communication with the CPU, the CPU can automatically detect and store the signal from the flow sensor to control sampling of exhaled breath. The CPU may further provide to the user/subject the appropriate alerts regarding prescribed time and dosage of the drug to be taken either based on pre-entered information or based on analysis of trends in drug blood concentration (that is determined based on the concentration of markers present in exhaled breath). Accordingly, it is contemplated herein that a MAMS of the subject invention can be portable.

According to the present invention, a data analyzer can compare a pattern of response (from the sensor) to previously measured and characterized responses from known markers. The matching of those patterns can be performed using a number of known techniques, including artificial intelligence systems (such as neural networks). By comparing analog output from a sensor (based on analyzed markers in the subject's exhaled breath sample) to a “blank” or control marker output using, for example, a neural network, a pattern can be established that is unique to that marker and the MAMS can subsequently learn to recognize the marker. In one embodiment, the artificial intelligence system can make an assessment of drug marker concentration and, based on the assessment, ascertain subject compliance with a prescribed drug regimen (including ascertaining whether the specific drug was taken by the subject and/or whether the appropriate drug dosage was taken by the subject). Where appropriate, the artificial intelligence system can also recommend an intervention (such as canceling, altering, maintaining, or adding to the prescription regimen) to ensure continued subject health and prevent drug diversion.

One conventional approach that can be used in a MAMS of the invention includes a neural network for processing data obtained from the sensor(s). As with most empirical modeling technologies, neural network development requires a collection of data properly formatted for use. Specifically, as known in the art, input data and/or the outputs of intermediate network processing layers may have to be normalized prior to use. It is known to convert the data to be introduced into a neural network into a numerical expression, to transform each of the numerical expressions into a number in a predetermined range, for example, by numbers between 0 and 1. Thus, the intelligence system of the present invention preferably has means for: 1) selecting at least a portion of the detected drug marker data from the sensor data output signal; 2) converting the selected portion of the detected drug marker data into numerical expressions; and 3) transforming the numerical expressions into a number in a predetermined range.

In accordance with one embodiment of the invention, the intelligence system is trained by providing to a neural network input data regarding expected levels/concentrations of the drug(s) marker in a sample of exhaled breath based on a prescribed regimen period or part thereof as well as output data from the sensors. The assessment by the intelligence system, along with the corresponding input data and output data is referred to as a data record. All available data records, possibly taken for a number of different subjects (such as male versus female; adult versus pediatric), comprise a data set. According to the present invention, a data set corresponding is stored in memory and is made available for use by the processing system for training, diagnostic and determinations. Normally, intelligence systems are trained ahead of time using data extracted from subjects. Using what is learned from the training data, the neural network may apply it to other/new subjects.

In one embodiment, the sensor's particular resistor geometries can be selected to optimize the desired response to a particular marker being sensed. For example, a self-calibrating polymeric “electronic nose” system is suitable for use in accordance with the subject invention to analyze either liquid or gas phase biological solutions for the presence and/or concentration of a target marker. In certain instances, the self-calibrating polymeric system is useful for detecting a variety of markers, and thus, a variety of drugs.

The results from MAMS analysis of the exhaled breath samples are optionally provided to the user (or subject) via a reporting means. In one embodiment, the sensor technology includes the reporting means. Contemplated reporting means include a computer processor linked to the sensor technology in which electronic or printed results can be provided. Alternatively, the reporting means can include a digital display panel, transportable read/write magnetic media such as computer disks and tapes which can be transported to and read on another machine, and printers such as thermal, laser or ink-jet printers for the production of a printed report.

The reporting means can provide the results to the user (or subject) via facsimile, electronic mail, mail or courier service, or any other means of safely and securely sending the report to the subject. Interactive reporting means are also contemplated by the present invention, such as an interactive voice response system, interactive computer-based reporting system, interactive telephone touch-tone system, or other similar system. The report provided to the user (or subject) may take many forms, including a summary of analyses performed over a particular period of time or detailed information regarding a particular sample analysis. Results may also be used to populate a laboratory database or a statistical database.

Preferably, in operation, the sensor will be used to identify a baseline spectrum for the subject prior to drug administration, if necessary. This will prove beneficial for the detection of more than one drug marker if the subject receives more than one drug at a time and possible interference from different foods and odors in the stomach, mouth, esophagus and lungs.

According to the present invention, a MAMS can be presented as a test kit for detecting the presence of target markers in a sample of exhaled breath, including: a housing; a sensor disposed within the housing, said sensor having the ability to detect the presence of and/or quantify the marker(s) in the exhaled breath sample; and a reporting module disposed within the housing adjacent to the sensor, said reporting module operatively connected to the sensor such that detection and/or quantification of the marker(s) by the sensor is communicated by the reporting module to the user.

In a related embodiment, a kit is provided for monitoring and controlling subject compliance with a drug regimen. In addition to a sensor, a breath sampling means, and reporting module, the kit can further include a drug dispenser and a dispenser control system, which is coupled to the dispenser. The dispenser control system allows for the controlled release of the drug to the subject based on the monitored subject compliance. Accordingly, the kit can further include a processing system coupled to the sensor, the reporting module, and the dispenser control system. The processing system preferably receives and analyzes input from the sensor(s) to determine subject compliance. The results generated by the processing system can be reported to the user with the reporting module. Where appropriate (such as those instances in which subject non-compliance is determined), the processing system can activate the dispenser control system to control the release of the drug to the subject.

Although MAMS is performed via sampling and analysis of exhaled breath samples, it is envisioned herein that MAMS can be equally effective in assessing subject compliance with bodily fluids (such as whole blood, blood plasma, urine, semen, saliva, lymph fluid, meningal fluid, amniotic fluid, glandular fluid, sputum, feces, sweat, mucous, and cerebrospinal fluid, including experimentally separated fractions of all of the preceding solutions or mixtures containing homogenized solid material, such as feces, tissues, and biopsy samples). The skilled artisan would readily acknowledge that the sensors described herein are also applicable to detecting drug markers in such bodily fluids.

### Breath Sampling

Generally, the exhalation gas stream comprises sequences or stages. At the beginning of exhalation there is an initial stage (Phase II), the gas representative thereof coming from an anatomically inactive (deadspace) part of the respiratory system, in other words, from the mouth and upper respiratory tracts. This is followed by a plateau stage (Phase III). Prior to the plateau stage, the gas is a mixture of deadspace and metabolically active gases. During the plateau phase, which comprises the last portion of the exhaled breath, nothing but deep lung gas, so-called alveolar gas is present. This gas, which comes from the alveoli, is termed end-tidal gas.

According to the present invention, exhaled gas from any specific phase of the respiratory cycle can be sampled to detect for the presence of target markers and/or quantify marker concentration in the sample of exhaled breath. For example, sensor technology as described herein can be applied to exhalation samples drawn from the initial phase, or the end-tidal (late plateau) phase.

Technology used for end-tidal component monitoring (such as CO₂ sensors, O₂ sensors, NO sensors, and thermistors) can be used to determine when or at what stage the exhaled breath sample is collected. Known methods for airway pressure measurements or for monitoring gas flow afford other means of collecting samples at the appropriate phase of the respiratory cycle. In a preferred embodiment, the exhaled breath sample is collected at end-tidal breathing.

In one embodiment, a VaporLab™ brand instrument is used to collect and analyze exhaled breath samples. The VaporLab™ instrument is a hand-held, battery powered SAW-based chemical vapor identification instrument suitable for detecting components in exhaled breath samples in accordance with the present invention. This instrument is sensitive to volatile and semi-volatile compounds using a high-stability SAW sensor array that provides orthogonal vapor responses for greater accuracy and discrimination. In a related embodiment, this instrument communicates with computers to provide enhanced pattern analysis and report generation. In a preferred embodiment, this instrument includes neural networks for “training” purposes, i.e., to remember chemical vapor signature patterns for fast, “on-the-fly” analysis.

In one example, a sensor of the subject invention would be used either in a clinical (healthcare) setting or remote subject-based location, to monitor appropriate delivery of drugs to a subject by detecting and/or measuring a target marker in subject exhaled breath that is generated from an additive administered concurrently with the drug.

One MAMS of the present invention is intended for use in a clinical setting (such as a hospital, a skilled nursing facility, a nursing home, and the like) where constant or semi-constant subject supervision is needed. In one embodiment, the MAMS is used for subjects requiring assisted ventilation. In this instance, the MAMS can be place “in-line” with the breathing circuit of a ventilator or other ventilation assist device. The breathing circuit can be any one of many conventional breathing circuits used in clinical settings for such purposes as assisted breathing, ventilation, anesthetic delivery, and the like. The breathing circuit sensor includes a sensor having a surface exposed to the gas stream and comprises a material selectively absorptive of a chemical vapor or group of vapors. The sensor is coupled to a computer having analyzing capabilities, where the sensor produces an electrical signal indicative of the presence of target markers in the vapors. The computer can be include further operative capabilities in determining the appropriate drug regimen of a particular subject, determining the approximate concentration of the target markers in the vapors, displaying results, signaling alarms, etc.

In another embodiment, the invention includes a method of monitoring a subject prior to, during, and after administration of a drug, wherein the subject is connected to the breathing circuit of a mechanical ventilator or other ventilation assist device. In the method, at least one sensor is exposed to a subject's expired gases prior to, during, and after administration of a drug to the subject; one or more target markers generated from the drug in situ is detected with the sensor(s); and the presence and/or concentration of the target marker is determined.

In yet another embodiment, the subject's exhaled breath is sampled and analyzed with a MAMS at the start of drug intervention (prior to administration of a drug) to formulate a baseline for comparison. For example, with markers that may be present in exhaled prior to drug administration, taking a baseline will ensure accurate assessment of drug compliance. Thus, establishing a baseline enables accurate reflection of subject compliance (or non-compliance) in taking a drug.

In a related embodiment, the invention includes a method of monitoring a subject prior to, during, and after administration of a gaseous drug (such as an anesthetic), wherein the subject is connected to a breathing circuit. In the method, a first sensor is exposed to inspired gases, wherein at least one inspired gas is a gaseous drug; a second sensor is exposed to expired gases; one or more target markers is detected with the sensors; and the presence and/or concentration of the target marker is determined.

In a further related embodiment, the method can also include the step of assessing the times at which the drug is delivered to the subject to ensure appropriate adherence to the drug regimen. An additional step to the method includes assessing whether appropriate adherence to the drug regimen has been performed; and recording and communicating the assessment regarding drug compliance.

In yet another embodiment, the invention includes an automated drug delivery and monitoring system for ensuring subject compliance in taking a prescribed drug. The automated system of the invention preferably automatically delivers appropriate drug dosages and specified times to a subject through a breathing circuit and/or an IV. According, the system includes: (1) a gaseous drug supply having a controller for controlling the amount of volatile drug provided by the supply to the breathing circuit; and/or (2) an IV drug supply having a controller for controlling the amount of IV drug administered to the subject intravenously; (3) an expired gas analyzer for analyzing the subject's breath for concentration of at least one marker indicative of the drug(s) presence and/or concentrations in the subject's bloodstream; and (4) a system controller connected to each of the drug supplies (IV and/or gaseous drug supplies), which receives the signal and controls the amount of drug administered via the breathing circuit and/or IV based on the signal. Where the system includes delivery of gaseous drug to the subject, the system preferably further comprises (5) an inspired gas analyzer for analyzing the concentration of gaseous drug in the breathing circuit.

Where a MAMS includes a breathing circuit, single or multiple samples collected by conventional in-line (or mainstream) sampling method are preferable, but if sensor acquisition time is reduced, side stream sampling may be used. With in-line sampling, a sensor of the subject invention is placed distal to the ET tube directly in the gas stream. In the latter, samples are collected through an adapter distal to the proximal end of an endotracheal (ET) tube and drawn through a thin bore tubing to a sensor of the subject invention. In certain embodiments that use in-line sampling, the sensor is placed in a sampling chamber positioned within the subject's gas stream. Alternatively to sample end-tidal gas, samples can be taken throughout the exhalation phase of respiration and an average value determined and correlated with blood concentration. Depending on the sample size and sensor response time, exhaled gas may be collected on successive cycles.

In a related embodiment, samples are collected at the distal end of an ET tube through a tube with a separate sampling port. This may improve sampling by allowing a “cleaner—(less deadspace)” sample during each respiratory cycle.

Certain embodiments of the subject invention provide sensor technologies that can quantify the concentration of markers present in an exhaled breath sample. Such systems and methods of the invention can further include reporting means for providing marker concentration results to the user (such as subject, clinician, pharmacist, and the like) for use in determining subject compliance and/or clinical applications (i.e., calculating the blood concentration of the drug in the subject). In a preferred embodiment, results from analysis can be communicated immediately upon sampling of exhaled breath. In related embodiments, such sensor technologies further include the use of a flow sensor to detect starting and completion of exhalation.

A useful construct of MAMS, as proposed in this application, is the ability to derive the subject's internal exposure to the drug, which is calculated from the concentration of drug marker present in exhaled breath samples and pre-existing knowledge of the drug's pharmacokinetic parameters. The computation of internal exposure allows one to estimate when the concentration of a drug in plasma drops below the so-called EC50, which is the commonly agreed-upon minimum concentration of drug in plasma for effectiveness.

Drug concentration in the subject (in particular, in the blood) as correlated with the concentration of markers in exhaled breath, may indicate when the subject is receiving a high dose (i.e., toxic dose), a low dose (i.e., ineffective dose), or effective (i.e., appropriate) dose of the drug. Knowledge of the exhaled breath marker concentration, and therefore the concentration (or dosage) of drug in blood, not only allows the user to monitor subject compliance in taking a drug, but also enables the user to know if the drug is accumulating in the blood, possibly leading to dangerously toxic levels of the drug, or that the concentration is falling, possibly leading to an inadequate dose of the drug. Monitoring changes in marker concentration in breath (and thus monitoring drug blood concentration) in accordance with the subject invention are, therefore, useful.

In certain embodiments, the subject invention enables the immediate monitoring of subject adherence to taking a drug. As contemplated herein, immediate monitoring refers to sampling and analysis of exhaled breath from a subject for target markers substantially completely within a short time period following administration of a drug (i.e., generally within a few minutes to about 24 hours).

In alternate embodiments, the subject invention enables deferred assessment of subject compliance in taking a drug. As contemplated herein, deferred assessment of subject compliance refers to sampling and analysis of exhaled breath from a subject after a certain amount of time has progressed, wherein the markers can still be detected in exhaled breath.

Accordingly, a system and/or method of the invention can be provided to a subject taking a drug for intermittent or continuous monitoring of drug adherence. In certain embodiments, the monitoring system and method of the subject invention can be administered to a subject taking a drug at prescribed intervals (such as on an hourly, daily, weekly, monthly, or even annual basis). Further, additional monitoring can be administered to a subject when an additional drug is prescribed. Also, concurrent monitoring for a plurality of prescribed drug regimens can also be performed using a MAMS of the invention.

Numerous variations of breath sampling apparatuses can be used to carry out the method of the present invention. For example, in one embodiment, the breath sampling apparatus includes a conventional flow channel through which exhalation air flows. The flow channel is provided with a sensor of the subject invention for detecting a target marker and/or measuring marker concentration. Furthermore, necessary output elements may be included with the breath sampling apparatus for delivering at least a measured concentration or detected marker result to the user, if necessary.

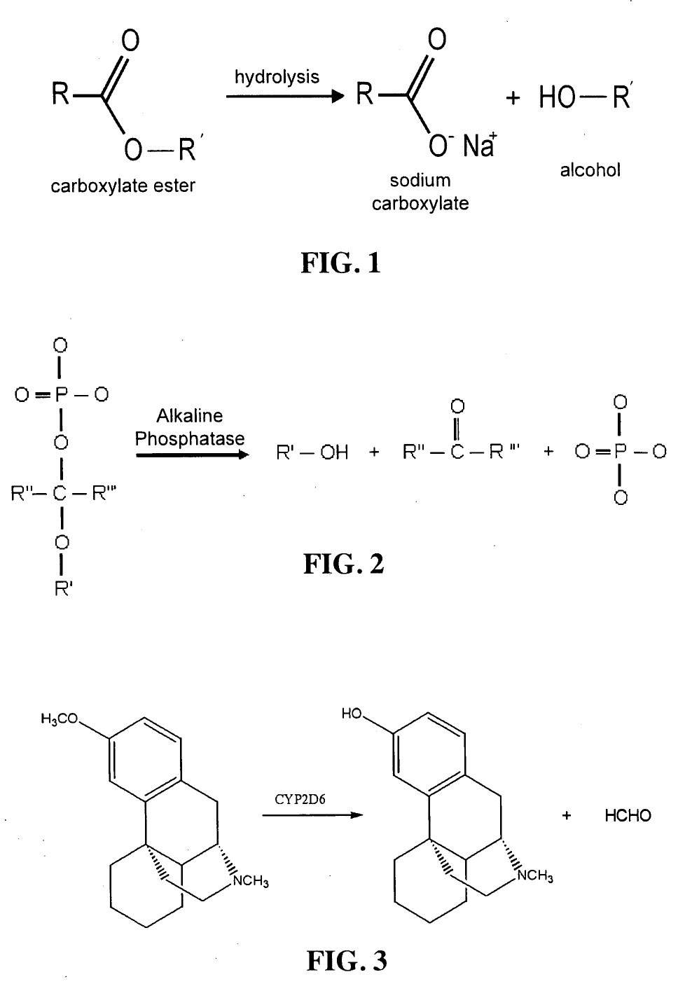

An alarm mechanism may also be provided. An instrument of similar type is shown in FIGS. 1 and 2 of U.S. Pat. No. 5,971,937.

In another embodiment, where the level of marker concentration in exhaled breath is measured, the marker concentration level is given a numerical value (for example, 50 on a scale of 1 to 100). Should the concentration fall below that value, the new value would be indicative of a decrease in concentration. Should the concentration increase beyond that value, the new value would be indicative of an increase in concentration. This numerical scale would allow for easier monitoring of changes in concentration. The numerical scale would also allow for easier translation into control signals for alarms, outputs, charting, and control of external devices (e.g., infusion pump). The upper and lower limits could be set to indicate thresholds such as from ineffective to dangerous drug levels.

The present invention contemplates the use of several collection devices designed to allow noninvasive collection of liquid phase components from exhaled breath, followed by one-step quantitative or semi-quantitative analysis of the condensate for the presence of and/or concentration of target markers. According to the present invention, the exhaled condensate may generally be collected via a mouthpiece held by the lips; however, in subjects with severe respiratory distress, the sample may be collected by fitting the subject with an airtight, snug-fitting facemask that allows the delivery of oxygen, while allowing the diversion of exhaled gases and liquid phase components into a condensate collection chamber such as those described below.

In general, the breath condensate collection devices of the invention comprise a collection chamber that has sterile, inner walls that can be cooled to a temperature sufficient to promote condensation of liquid phase components from gaseous phase components in exhaled breath. Preferably, the inner walls of the collection chamber can be cooled to a temperature at about or below 32° F. Such breath condensate collection devices are preferably disposable and lightweight.

In certain embodiments, the condensate collection devices of the invention include coaxial chambers with an interposed area containing coolant that can be chilled externally or via an internal endothermic reaction. Such breath condensate collection devices are well-known and currently available. Examples of such devices include those generally described in U.S. patent application Ser. Nos. 10/42,721 and 10/778,477.

In one embodiment, a device for collecting and analyzing liquid phase components of exhaled breath from a subject includes: an expiratory flow tube that serves as a conduit for sampling exhaled breath of the subject and a breath condensate collection device. In one embodiment of the invention, the condensate collection device comprises: a central chamber having an interior, wherein said central chamber may be cooled to a temperature sufficient to promote condensation of the liquid phase components from the gaseous components in exhaled breath (for example, at about 32° F. and below); a breath input assembly, disposed at one end of the central chamber, in fluid communication with the interior of the central chamber and the expiratory flow tube, whereby the breath input assembly connects the expiratory flow tube and the central chamber; an exit assembly, disposed at the other end of the central chamber, in fluid communication with the interior of the central chamber; and a vacuum device connected to the exit assembly for collecting condensated liquid components of exhaled breath from the central chamber.

In another embodiment of the present invention, a breath condensate collection device includes: a central chamber having an interior and first and second opposing ends; a breath input assembly in fluid communication with the interior of the central chamber; and an exit assembly in fluid communication with the interior of the central chamber, wherein the exit assembly includes a narrow tube, said narrow tube having a sensor disposed therein, said sensor having the ability to detect with high specificity the presence and/or concentration of a target marker.

In features of this aspect, the sensor is a fiber matrix impregnated with aptamers specific for a target marker; and the device further includes a plunger assembly having a piston and a handle, wherein the piston is slidably disposed in the interior of the central chamber and wherein the handle extends from the first end of the central chamber so as to permit the piston to be moved within the central chamber, whereby the collected breath condensate may contact the fibrous aptamer matrix disposed within the narrow tube. In certain related embodiments, the condensate collection device further includes a viewing window, through which visible detection of a physical, visible change in aptamer binding to a target marker can be performed.

In yet another embodiment, the sensor disposed in the narrow tube comprises functionalized aptamers attached to a gold plated sensor, where the free end of the aptamer is functionalized with methylene blue. In the presence of the target marker, the aptamer bends and the methylene blue end contacts the gold plated sensor, changing the current flow to communicate to the user the presence of the target marker. See, for example, Xiao Y et al., “A reagentless signal-on architecture for electronic, aptamer-based sensors via target-induced strand displacement,” J Am Chem Soc., 127(51):17990-1 (2005); Xiao Y et al., “Label-free electronic detection of thrombin in blood serum by using an aptamer-based sensor,” Angew Chem Int Ed Engl., 26; 44(34):5456-9 (2005); Jhaveri, S. et al., “In vitro selection of signaling aptamers,” Nat Biotechnol., 18(12):1293-7 (2000); and Collett J R et al., “Production and processing of aptamer microarrays,” Methods, 37(1):4-15 (Epub 2005 Sep. 30)).

### Sensor Technology

According to the subject invention, any one of the many commercially available off the shelf (COTS)-based analytical approaches for measurement of analytes in gaseous and/or liquid phase mediums can be used to detect and/or quantify markers in exhaled breath. It is contemplated that the MAMS of the invention may comprise at least one sensor, or a plurality of sensors, for capturing the desired marker concentration data. Each sensor generates an output signal based on the presence of the drug marker(s) in a sample of exhaled breath (or bodily fluid). Examples of certain COTS-based approaches that can be used in accordance with the systems and methods described herein include, but are not limited to, high electron mobility transistors (HEMT), nuclear magnetic resonance (NMR), polymer based membranes—chemoresistive (Cyranose); polymer-surface acoustic wave (SAW) and electrochemical chemical array (Hazmatcad and Hazmatcad Plus); spectroscopy-based analysis; visible spectroscopy; UV spectroscopy; TIF TIFXP-1A Negative Corona Leak Detector; negative ion capture sensors; heated sensors/ceramic semiconductor sensors; infrared absorption; nuclear magnetic resonance spectroscopy; photoemission spectroscopy; Raman spectroscopy; Fourier transform spectroscopy—FTIR; time-resolved spectroscopy; flame spectroscopy; plasma emission spectroscopy; force spectroscopy; dielectric spectroscopy; circular dichroism spectroscopy; refractory indices; and the like. Other contemplated sensors include sensors based on microcantilevers, molecularly imprinted polymers, and amplifying fluorescent polymers. In a preferred embodiment, small scale gas chromatography sensor technology is used in accordance with the subject invention.

The invention preferably utilizes sensor technology, such as commercial devices known as “artificial” or “electronic” tongues or noses, to non-invasively monitor marker presence and/or concentration in exhaled breath. Electronic noses have been used mostly in the food, wine, and perfume industry where their sensitivity makes it possible to distinguish between odorous compounds. For example, electronic noses have been useful in distinguishing between grapefruit oil and orange oil in the perfume industry and in identifying spoilage in perishable foods before the odor is evident to the human nose.

In the past, there was little medical-based research and application of these artificial/electronic tongues and noses. However, recent use has demonstrated the power of this non-invasive technique. For example, electronic noses have been used to determine the presence of bacterial infection in the lungs by analyzing the exhaled gases of subjects for odors specific to particular bacteria (Hanson C W, Steinberger H A, “The use of a novel electronic nose to diagnose the presence of intrapulmonary infection,” Anesthesiology, 87(3A):Abstract A269, (1997)). Also, a genitourinary clinic has utilized an electronic nose to screen for and detect bacterial vaginosis, with a 94% success rate after training (Chandiok S, et al., “Screening for bacterial vaginosis: a novel application of artificial nose technology,” Journal of Clinical Pathology, 50(9):790-1 (1997)). Specific bacterial species can also be identified with the electronic nose based on special odors produced by the organisms (Parry A D et al., “Leg ulcer odor detection identifies beta-haemolytic streptococcal infection,” Journal of Wound Care, 4:404-406 (1995)).

A number of patents that describe gas sensor technology that can be used in the subject invention include, but are not limited to, the following: U.S. Pat. Nos. 5,945,069; 5,918,257; 4,938,928; 4,992,244; 5,034,192; 5,071,770; 5,145,645; 5,252,292; 5,605,612; 5,756,879; 5,783,154; and 5,830,412. Other sensors suitable for the present invention include, but are not limited to, metal-insulator-metal ensemble (MIME) sensors, cross-reactive optical microsensor arrays, fluorescent polymer films, surface enhanced raman spectroscopy (SERS), diode lasers, selected ion flow tubes, metal oxide sensors (MOS), non-dispersive infrared spectrometer, bulk acoustic wave sensors, calorimetric tubes, functionalized microcantilevers, and infrared spectroscopy.

Recent developments in the field of detection that can also be used as sensors for the subject invention include, but are not limited to, gas chromatography, semiconductive gas sensors, mass spectrometers (including proton transfer reaction mass spectrometry), and infrared (IR) or ultraviolet (UV) or visible or fluorescence spectrophotometers (i.e., non-dispersive infrared spectrometer). For example, with semiconductive gas sensors, markers cause a change in the electrical properties of semiconductor(s) by making their electrical resistance vary, and the measurement of these variations allows one to determine the concentration of marker(s). In another example, gas chromatography, which consists of a method of selective detection by separating the molecules of gas compositions, may be used as a means for analyzing markers in exhaled breath samples.

In accordance with the subject invention, sensors for detecting/quantifying markers utilize a relatively brief detection time of around a few seconds. Other recent gas sensor technologies contemplated for use in a MAMS of the present invention include apparatuses that utilize conductive-polymer gas-sensors (“polymeric”), aptamer biosensors, amplifying fluorescent polymer (AFP) sensors, or apparatuses having surface-acoustic-wave (SAW) gas-sensors.

Conductive-polymer gas-sensors (also referred to as “chemoresistors”) have a film made of a conductive polymer sensitive to molecules of target (sometimes odorous) substances. Upon contact with target marker molecules, the electric resistance of the sensors changes, which provides an indication of the marker's presence. The measurement of the variation of this resistance enables determination of the concentration of the markers present. An advantage of this type of sensor is that it functions at temperatures close to room temperature. Different sensitivities for detecting different markers can be obtained by modifying or choosing an alternate conductive polymer.

Polymeric gas sensors can be built into an array of sensors, where each sensor is designed to respond differently to different markers and augment the selectivity of the drug markers. For example, a sensor of the subject invention can comprise of an array of polymers, (i.e., 32 different polymers) each exposed to a marker. Each of the individual polymers swells differently to the presence of a specific marker, creating a change in the resistance of that membrane and generating an analog voltage in response to that specific marker (“signature”). The normalized change in resistance can then be transmitted to a processor to identify the type, quantity, and/or quality of the marker based on the pattern change in the sensor array. The unique response results in a distinct electrical fingerprint that is used to characterize the marker. The pattern of resistance changes of the array is diagnostic of the marker in the sample, while the amplitude of the pattern indicates the concentration of the marker in the sample.

Responses of polymeric gas sensors to target markers can be fully characterized using a combination of conventional gas sensor characterization techniques. For example, the sensor can be attached to a computer. The results can be displayed on the computer screen, stored, transmitted, etc. A data analyzer can compare a pattern of response to previously measured and characterized responses from known substances. The matching of those patterns can be performed using a number of techniques, including neural networks. By comparing the analog output from each of the 32 polymers to a “blank” or control, for example, a neural network can establish a pattern that is unique to that marker and subsequently learns to recognize that marker. The particular resistor geometries are selected to optimize the desired response to the particular marker being sensed. In one embodiment, the sensor of the present invention is a self-calibrating polymer system suitable for liquid or gas phase biological solutions for detecting a variety of target markers simultaneously.

Another sensor of the invention can be provided in the form of an aptamer. In one embodiment, the SELEX™ (Systematic Evolution of Ligands by EXponential enrichment) methodology is used to produce aptamers that recognize drug markers with high affinity and specificity. Aptamers produced by the SELEX methodology have a unique sequence and the property of binding specifically to a desired marker. The SELEX methodology is based on the insight that nucleic acids have sufficient capacity for forming a variety of two- and three-dimensional structures with sufficient chemical versatility available within their monomers to act as ligands (form specific binding pairs) for virtually any chemical compound, whether monomeric or polymeric.

According to the subject invention, drug markers of any size or composition can thus serve as targets for aptamers. See also Jayasena, S., “Aptamers: An Emerging Class of Molecules That Rival Antibodies for Diagnostics,” Clinical Chemistry, 45:9, 1628-1650 (1999).

Aptamer biosensors can be utilized in the present invention for detecting the presence of markers in exhaled breath samples. In one embodiment, aptamer-based sensors are composed of resonant oscillating quartz sensors that can detect minute changes in resonance frequencies due to modulations of mass of the oscillating system, which results from a binding or dissociation event of an aptamer to a target marker.

Similarly, molecular beacons (MB) and molecular beacon aptamers (MBA) employ fluorescence resonance energy transfer based methods to provide fluorescence signal increases in the presence of particular target sequences. Essentially, molecular beacons are attached to natural or synthetic ligands (such as aptamers, enzymes, antibodies, etc.), where upon binding of the ligand to a target marker, the molecular beacon generates a signal that is visibly detectable by the user. See also, Stojanovic, Milan N., de Prada, Paloma, and Landry, Donald W., “Aptamer-Based Folding Fluorescent Sensor for Cocaine” J. Am. Chem. Soc. 2001, 123, 4928-4931 (2001); Jayasena, Sumedha D., “Aptamers: An Emerging Class of Molecules That Rival Antibodies of Diagnostics, Clinical Chemistry 45:9, 1628-1650 (1999).

Amplifying fluorescent polymer (AFP) sensors may be utilized in the present invention for detecting the presence of drug markers in exhaled breath samples. AFP sensors are extremely sensitive and highly selective chemosensors that use amplifying fluorescent polymers. When target markers bind to thin films of the polymers, the fluorescence of the film decreases. A single molecule binding event quenches the fluorescence of many polymer repeat units, resulting in an amplification of the quenching. The binding of markers to the film is reversible, therefore the films can be reused.

Surface-acoustic-wave (SAW) sensors oscillate at high frequencies and generally have a substrate, which is covered by a chemoselective material. In SAW sensors, the substrate is used to propagate a surface acoustic wave between sets of interdigitated electrodes (i.e., to form a transducer). The chemoselective material is coated on the transducer. When a marker interacts with the chemoselective material coated on the substrate, the interaction results in a change in the SAW properties, such as the amplitude of velocity of the propagated wave. The detectable change in the characteristic wave is generally proportional to the mass load of the marker(s) (i.e., concentration of the marker in exhaled breath, which corresponds to the concentration of the drug in the subject's blood stream).

Certain embodiments of the invention use known SAW devices, such as those described in U.S. Pat. Nos. 4,312,228 and 4,895,017, and Groves W. A. et al., “Analyzing organic vapors in exhaled breath using surface acoustic wave sensor array with preconcentration: Selection and characterization of the preconcentrator adsorbent,” Analytica Chimica Acta, 371:131-143 (1988). Other types of chemical sensors known in the art that use chemoselective coating applicable to the manufacture and operation of a MAMS of the present invention include bulk acoustic wave (BAW) devices, plate acoustic wave devices, interdigitated microelectrode (IME) devices, optical waveguide (OW) devices, electrochemical sensors, and electrically conducting sensors.

In one embodiment, the sensor of the invention is based on surface acoustic wave (SAW) sensors. The SAW sensors preferably include a substrate with piezoelectric characteristics covered by a polymer coating, which is able to selectively absorb target markers. SAW sensors oscillate at high frequencies and respond to perturbations proportional to the mass load of certain molecules. This occurs in the vapor phase on the sensor surface.

In a related embodiment, a MAMS of the invention uses a sensor based on a SAW sensor of Stubbs, D. et al. (see Stubbs, D. et al., “Investigation of cocaine plumes using surface acoustic wave immunoassay sensors,” Anal Chem., 75(22):6231-5 (November 2003) and Stubbs, D. et al., “Gas phase activity of anti-FITC antibodies immobilized on a surface acoustic wave resonator device,” Biosens Bioelectron, 17(6-7):471-7 (2002)). For example, the sensor of the subject invention can include a two-port resonator on ST-X quartz with a center frequency of 250 MHz. On the cut quartz, a temperature compensated surface acoustic wave (SAW) is generated via an interdigital transducer. Antibodies specific to a target marker are then attached to the electrodes (i.e., 1.5 micron wide) on the sensor device surface via protein cross linkers. In the vapor phase on the sensor surface, when target markers are present, a change in frequency occurs to alert the user that a target marker has been recognized.

In a related embodiment, the SAW sensor is connected to a computer, wherein any detectable change in frequency can be detected and measured by the computer. In a preferred embodiment, an array of SAW sensors (4-6) is used, each coated with a different chemoselective polymer that selectively binds and/or absorbs vapors of specific classes of molecules. The resulting array, or “signature” identifies specific compounds.

The operating performance of most chemical sensors that use a chemoselective film coating is greatly affected by the thickness, uniformity and composition of the coating. For these sensors, increasing the coating thickness, has a detrimental effect on the sensitivity. Only the transducer senses the portion of the coating immediately adjacent to the transducer/substrate.

For example, if the polymer coating is too thick, the sensitivity of a SAW device to record changes in frequency will be reduced. These outer layers of coating material compete for the marker with the layers of coating being sensed and thus reduce the sensitivity of the sensor. Uniformity of the coating is also a critical factor in the performance of a sensor that uses a chemoselective coating since changes in average surface area greatly affect the local vibrational signature of the SAW device. Therefore, films should be deposited that are flat to within 1 nm with a thickness of 15-25 nm. In this regard, it is important not only that the coating be uniform and reproducible from one device to another, so that a set of devices will all operate with the same sensitivity, but also that the coating on a single device be uniform across the active area of the substrate.

If a coating is non-uniform, the response time to marker exposure and the recovery time after marker exposure are increased and the operating performance of the sensor is impaired. The thin areas of the coating respond more rapidly to a target marker than the thick areas. As a result, the sensor response signal takes longer to reach an equilibrium value, and the results are less accurate than they would be with a uniform coating.

Most current technologies for creating large area films of polymers and biomaterials involve the spinning, spraying, or dipping of a substrate into a solution of the macromolecule and a volatile solvent. These methods coat the entire substrate without selectivity and sometimes lead to solvent contamination and morphological inhomogeneities in the film due to non-uniform solvent evaporation. There are also techniques such as microcontact printing and hydrogel stamping that enable small areas of biomolecular and polymer monolayers to be patterned, but separate techniques like photolithography or chemical vapor deposition are needed to transform these films into microdevices.

Other techniques such as thermal evaporation and pulsed laser ablation are limited to polymers that are stable and not denatured by vigorous thermal processes. More precise and accurate control over the thickness and uniformity of a film coating may be achieved by using pulsed laser deposition (PLD), a physical vapor deposition technique that has been developed recently for forming ceramic coatings on substrates. By this method, a target comprising the stoichiometric chemical composition of the material to be used for the coating is ablated by means of a pulsed laser, forming a plume of ablated material that becomes deposited on the substrate.

Polymer thin films, using a new laser based technique developed by researchers at the Naval Research Laboratory called Matrix Assisted Pulsed Laser Evaporation (MAPLE), have recently been shown to increase sensitivity and specificity of chemoselective Surface Acoustic Wave vapor sensors. By providing improved SAW biosensor response by eliminating film imperfections induced by solvent evaporation and detecting molecular attachments to specific target markers, high sensitivity and specificity is possible.

Certain extremely sensitive, commercial off-the-shelf (COTS) electronic noses, such as those provided by Cyrano Sciences, Inc. (“CSI”) (i.e., CSI's Portable Electronic Nose and CSI's Nose-Chip integrated circuit for odor-sensing, see U.S. Pat. No. 5,945,069—FIG. 1), may be used in the system and method of the present invention to monitor the exhaled breath from a subject. These devices offer minimal cycle time, can detect multiple markers, can work in almost any environment without special sample preparation or isolation conditions, and do not require advanced sensor design or cleansing between tests.

In other embodiments, competitive binding immunoassays can be used to test a bodily fluid sample for the presence of signaling agents. Immunoassay tests generally include an absorbent, fibrous strip having one or more reagents incorporated at specific zones on the strip. The bodily fluid sample is deposited on the strip and by capillary action the sample will migrate along the strip, entering specific reagent zones in which a chemical reaction may take place. At least one reagent is included which manifests a detectable response, for example a color change, in the presence of a minimal amount of a signaling agent of interest. Patents that describe immunoassay technology include the following: U.S. Pat. Nos. 5,262,333 and 5,573,955.

In one embodiment, the device of the present invention may be designed so that subjects can exhale via the mouth or nose directly onto a sensor of the invention, without needing a breath sampling apparatus. For example, a mouthpiece or nosepiece will be provided for interfacing a subject with the device to readily transmit the exhaled breath to the sensor (See, i.e., U.S. Pat. No. 5,042,501). In a related embodiment, wherein the sensor is connected to a neural network, the output from the neural network is similar when the same subject exhales directly into the device and when the exhaled gases are allowed to dry before the sensor samples them.

In another embodiment, a subject's breath sample can be captured in a container (vessel) for later analysis using a sensor of the subject invention (i.e., mass spectrometer).

The humidity in the exhaled gases represents a problem for certain electronic nose devices (albeit not SAW sensors) that only work with “dry” gases. When using such humidity sensitive devices, the present invention may adapt such electronic nose technology so that a subject can exhale directly into the device with a means to dehumidify the samples. This is accomplished by including a commercial dehumidifier or a heat moisture exchanger (HME), a device designed to prevent desiccation of the airway during ventilation with dry gases.

Alternatively, the subject may exhale through their nose, which is an anatomical, physiological dehumidifier to prevent dehydration during normal respiration. Alternatively, the sensor device can be fitted with a preconcentrator, which has some of the properties of a GC column. The gas sample is routed through the preconcentrator before being passed over the sensor array. By heating and volatilizing the gases, humidity is removed and the marker being measured can be separated from potential interferents.

### Remote Communication System

A further embodiment of the invention includes a communications device in the home (or other remote location) that will be interfaced to a MAMS of the invention. The home communications device will be able to transmit immediately or at prescribed intervals directly or over a standard telephone line (or other communication transmittal means) the data collected by the MAMS of the invention. The communication of the data will allow the user (i.e., physician) to be able to remotely verify if the subject has complied in taking a give drug and/or if the appropriate dosage of a drug is being administered to the subject.

The data transmitted from the home can also be downloaded to a computer where the detected presence of the marker and/or drug blood levels are stored in a database, and any deviations outside of the stored data is flagged so that a user could be notified of subject adherence. In one embodiment, the downloaded information pertains to drug marker levels/concentration (or even calculated drug blood levels based on detected marker levels in breath) and deviations outside of a given concentration (thus pharmacological efficacy of the drug) would be automatically flagged (i.e., alarm) so that a user (i.e., subject, physician, nurse) could appropriately adjust the drug dosage per suggestions provided by a computer processing unit connected to the sensor or per dosage suggestions provided by health care personnel (i.e., physician).

In view of the above, the present invention provides the capability of non-invasively, and in certain instances continuously, monitoring subject compliance in taking a wide variety of drugs, using exhaled breath as a surrogate.

### Drug Markers

In accordance with the present invention, drug markers that are useful as an indication of drug presence and/or concentration in the subject include the following olfactory markers, without limitation: dimethyl sulfoxide (DMSO), acetaldehyde, acetophenone, trans-Anethole (1-methoxy-4-propenyl benzene) (anise), benzaldehyde (benzoic aldehyde), benzyl alcohol, benzyl cinnamate, cadinene, camphene, camphor, cinnamaldehyde (3-phenylpropenal), garlic, citronellal, cresol, cyclohexane, eucalyptol, and eugenol, eugenyl methyl ether; butyl isobutyrate (n-butyl 2, methyl propanoate) (pineapple); citral (2-trans-3,7-dimethyl-2,6-actadiene-1-al); menthol (1-methyl-4-isopropylcyclohexane-3-ol); and α-Pinene (2,6,6-trimethylbicyclo-(3,1,1)-2-heptene). These markers are preferred since they are used in the food industry as flavor ingredients and are permitted by the Food and Drug Administration. As indicated above, olfactory markers for use in the present invention can be selected from a vast number of available compounds (see Fenaroli's Handbook of Flavor Ingredients, 4^{th} edition, CRC Press, 2001) and use of such other applicable markers is contemplated herein.

The markers of the invention also include compounds that have been federally approved and categorized as GRAS (“generally recognized as safe”), which are available on a database maintained by the U.S. Food and Drug Administration Center for Food Safety and Applied Nutrition. Markers categorized as GRAS that are readily detectable in exhaled breath include, but are not limited to, sodium bisulfate, dioctyl sodium sulfosuccinate, polyglycerol polyricinoleic acid, calcium casein peptone-calcium phosphate, botanicals (i.e., chrysanthemum; licorice; jellywort, honeysuckle; lophatherum, mulberry leaf; frangipani; selfheal; sophora flower bud), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

Halogenated compounds (i.e. fluorinated drugs or markers) hold particular promise as they are readily highly volatile, safe for human consumption, and are readily detected in exhaled breath with portable Freon leak detectors. Some of these compounds are used as propellants for delivery of drugs via the pulmonary route, such as metered dose inhalers and therefore are known to be safe and are FDA approved, some are GRAS compounds as well. The technologies most often used to detect Freon leaks include: Negative Ion Capture, Heated Sensor/Ceramic Semiconductor, Infrared Absorption, and TIF TIFXP-1A Negative Corona Leak Detector. Many drugs are fluorinated and metabolites are often extremely volatile and detectable in exhaled breath. Numerous such compounds are available that could be used as markers and could be added as excipients during the manufacture of drugs.

As described above, markers are detected by their physical and/or chemical properties. drug markers, as contemplated herein, are by-products derived from additives that are added to a desired drug regimen to enhance differentiation in detection/quantification of the markers in exhaled breath. As described herein, drug markers are detected in exhaled breath upon absorption, distribution, metabolism, and/or excretion of the additives by a subject. Generally, in accordance with the present invention, drug markers are poorly soluble in water, which enhances their volatility and detection in the breath.

According to the subject invention, the additives to be combined with a drug for ease of marker detection in exhaled breath can have any one or combination of the following characteristics: (1) applicability to all orally administered drugs (for example, drugs administered orally either once—PO Q or twice—PO BID per day) that are used in clinical medicine (meaning the additive by-product or marker detected in the breath is not related to the active pharmaceutic/drug or one its metabolites); (2) applicability to QD or BID dosing, where the duration of generated marker presence in exhaled breath is not greater than 5 hours or less than 1 hours; (3) no limitation on the type of metabolism that enables generation of markers in exhaled breath from additives (for example, non-CYP metabolism (e.g., esterase) to avoid potential drug-drug interactions (DDIs)); (4) the enzyme system that acts upon the additive to release a detectable marker in exhaled breath should not be susceptible to a high incidence of genetic variability, should not suffer from a high incidence of drug-induced inhibition of function (adverse drug reactions), and should have sufficient catalytic capacity to generate the marker from the additive even in the face of factors that can lower its functional activity; (5) the marker generated from the additive is metabolically stable; (6) additive presence with drug does not alter either the pharmacodynamics (PD) or the pharmacokinetics (PK) of the drug (e.g., same bioequivalence, same metabolism); (7) inexpensive, readily available, and easy to synthesize; (8) easy to integrate as an excipient into primary drug tablets (or other formulation for administration); and (9) non-flammable; imposes no physical danger on active drug during manufacturing or while the subject stores the drug.

According to the subject invention, the markers generated from the additives combined with a drug can have any one or combination of the following characteristics: (1) no intrinsic toxicity at concentrations required for MAMS utility; (2) is a generally recognized as safe (GRAS) compound; (3) is an FDA approved chemical entity; (4) is a compound that is not approved by the FDA per se for purposes but whose toxicology data in humans can be used to support regulatory approval; and (5) is a new chemical entity (NCE) having no toxicological data in humans; (6) is unique in the breath (e.g., not found in multiple foods or not endogenously generated), where the marker provides an outstanding signal to noise (S:N) ratio with a MAMS sensor of the invention, and does not require a baseline MAMS reading; (7) has rapid onset of appearance in the breath after additive/drug absorption by the subject; (8) has a reproducible duration of appearance in the breath; (9) is easily detectable by multiple sensor technologies that are rapid, portable, inexpensive, compact, and available for point-of-care (POC) analysis; (10) is present in breath in sustainable concentrations at oral additive doses that are not excessive to put into tablets as excipients (for example, the dose of additive-excipient combined with the active drug in a pill cannot be excessive and must generate enough of the marker so that the marker can be detected in the breath using a sensor device); and (11) marker is generated from a flexible chemistry formulation platform that will allow the selection of optimal markers with regards to time of presence in the breath for MAMS application.

According to the subject invention, upon concurrent administration of a drug and an additive, the detection of the marker (such as the metabolite of the additive) can occur under several circumstances. In one example where the drug is administered orally, the marker can “coat” or persist in the mouth, esophagus and/or stomach upon ingestion and be detected with exhalation (similar to the taste or flavor that remains in the mouth after eating a breath mint).

In a second instance where the drug and additive are administered orally, the additive may react with the environment in the mouth or stomach to produce or liberate the marker that can then be detected upon exhalation (for example, non-enzymatic reactions in the mouth can cause the release of the marker in exhaled breath; acids or enzymes may also produce or liberate the marker). Thirdly, the additive can be absorbed in the gastrointestinal tract and be excreted in the lungs (i.e. alcohol is rapidly absorbed and detected with a Breathalyzer). Generally, a drug marker of the invention provides a means for determining not only subject compliance in taking a drug but can also be used for determining the pharmacodynamics and pharmacokinetics of the drug (based on correlation of marker concentration in breath with drug concentration in blood).

In one embodiment, an additive is concurrently administered with a drug (i.e., additive is provided in a pharmaceutically acceptable carrier, additive is provided in drug coating composed of rapidly dissolving glucose and/or sucrose) in the form of a pill. For drugs administered in the form of pills, capsules, and fast-dissolving tablets, the additives can be applied as coatings or physically combined or added to drug. Additives can also be included with drugs that are administered in liquid form (i.e., syrups, via inhalers, or other dosing means).

In one embodiment, the additive would not be susceptible to degradation by enzymes located in the mouth (saliva). Specifically, a devious subject (e.g., schizophrenic, subject who has a court ordered drug therapy) or cognitively impaired subject (e.g., Alzheimer's subject) could intentionally or unintentionally “chew” the an oral drug (such as a tablet or capsule) in his/her mouth and generate the target marker in the oral cavity (via action by salivary enzymes), which in turn may be detected using a sensor of the invention and thus generate a false positive indication of pill ingestion.

In certain embodiments, even with the chewing of an oral drug (such as a tablet) containing an additive that is potentially susceptible to degradation by mouth enzymes, it is likely that the maximum concentration of the target marker (TM CMax) and the target marker concentration-time relationships would be markedly different than those marker concentrations generated by the action of enzymes located in the gut, blood and/or liver (for example, relative to gut, blood and/or liver enzymes, mouth enzymes in the setting of tablet chewing would generate a much lower TM CMax in breath and have a flattened target marker concentration-time relationship with a lower area under the curve (AUC). Such characteristics should allow for identification of subject deviousness or impairment in adhering to proper drug compliance.

As noted above, in some embodiments, the target marker can include a volatile organic compound (VOC) that is either naturally or non-naturally occurring in the body, such as formaldehyde. For example, a VOC metabolite can be the product of enzyme action (e.g., CYP metabolism) for a number of drugs, including but not limited to dextromethorphan and verapamil.

The drug markers of the invention could be used for indicating specific drugs or for a class of drugs. For example, a subject may be taking an anti-depressant (tricyclics such as nortriptyline), antibiotic, an antihypertensive agent (i.e., beta-blocker, angtiotensin converting enzyme (ACE) inhibitor; angiotensin receptor blocker (ARB); pain drug; and (esophageal) anti-reflux drug). One marker could be used for antibiotics as a class, or for subclasses of antibiotics, such as erythromycins. Another marker could be used for antihypertensives as a class, or for specific subclasses of antihypertensives, such as calcium channel blockers. The same would be true for the anti-reflux drug. Furthermore, combinations of marker substances could be used allowing a rather small number of markers to specifically identify a large number of drugs.

### Strategies for Designing Markers

According to the subject invention, various design considerations can be taken into account when developing a marker. Such considerations include, but are not limited to: (1) the use of different biological gating mechanisms to generate the marker in exhaled breath such as, but not limited to, enzyme-based mechanisms; environmental-based mechanisms (for example, bodily functions such as stomach pH to induce pH mediated hydrolysis of additive to release a detectable marker); this is especially useful for many subjects on H2 receptor antagonists (e.g., cimetidine, ranitidine) or PPIs (e.g., lansoprazole, omeprazole, pantoprazole, and esomeprazole), which can markedly increase pH in the subject; (2) the of different biological absorption mechanisms at different biological sites such as, but not limited to, the stomach; intestine; liver; and blood; (3) the use of different phases of detection media from breath that could be used to detect a marker such as, but not limited to, gas phase measurements in breath and liquid phase in condensate of breath (exhaled breath condensate—EBC); and (4) the use of different methods to gate the release of the marker to be measured in breath such as, but not limited to, slow release of a very short lived volatile agent based on environmental factors (e.g., slow release of drug on pH-gated drug release) or slow release of a very short lived volatile agent based on slow enzymatic breakdown of an additive that is a prodrug (e.g., esterase cleaves A→B).

In one embodiment of the invention, different types of additives and markers are identified that can be measured using a sensor/COTS device meeting the criterion established above. In other words, the additive or marker of the invention is selected based on which sensing technology will meet the criteria for detecting the marker in exhaled breath. In a preferred embodiment, a COTS-based MAMS is selected for use in detecting a marker in exhaled breath. In other embodiments, a nano-based sensor is selected for use in detecting a marker in exhaled breath.

In one embodiment of the invention, the additive that is ingested with a drug, which may or may not be the marker itself, is created from any one of the following: Class I agents (GRAS compounds), Class II agents (FDA approved chemical entities), Class III agents (compounds not approved by the FDA for purposes but whose toxicology data in humans can be used to support regulatory approval); or Class IV agents (NCEs having no toxicological data in humans) as chemical templates to generate an additive.

When contemplating the various classes of agents for use as markers in accordance with the present invention, the following types of compounds can be considered: aldehydes; alcohols; ketones; enols; ethers; esters; and phosphate-containing compounds. Esters are particularly attractive as markers for the purposes of the present invention because many are already used in the food and/or perfume industry. Examples of esters for use as markers in accordance with the present invention include, but are not limited to, methyl butanoate (pineapple or apple); methyl salicylate (oil of wintergreen); methyl benzoate (marzipan); ethyl butanoate (pineapple); ethyl methanoate (raspberry or rum); ethyl butanoate (pineapple or apricot or strawberry); ethyl salicylate (mint); ethyl heptanoate (grape); butyl ethanoate (raspberry); pentyl ethanoate (banana); pentyl pentanoate (apple); pentyl butanoate (pear or apricot); octyl ethanoate (orange); and benzyl ethanoate jasmine).

In one embodiment of the invention, a marker is selected based on the ability of an additive to be sustainably released into the gastrointestinal tract or blood. In certain related embodiments, where the additive is the marker, the additive is relatively volatile and can exit the body unchanged (no metabolism). In contrast, where the marker is generated from the additive, the slow release of the additive from its storage site will rapidly generate a marker that is detectable in exhaled breath.

In another embodiment of the invention, additives are developed that are a type of pro-“marker”-drug (e.g., ester compounds), which have progressively higher degrees of steric/electronic hindrance in the structure (less able for enzymes such as esterases or alkaline phosphatase, for example, to cleave the molecule), that have a wide range of half life in blood and thus a wide range of durations of marker release. In this manner, an additive pro-“marker”-drug is custom designed to slowly release the marker identified in the breath.

### Drugs

As contemplated herein, drugs to be monitored in accordance with the subject invention include, but are not limited to, anesthetic agents, psychiatric drugs (i.e., antidepressants, anti-psychotics, anti-anxiety drugs, depressants), analgesics, stimulants, biological response modifiers, NSAIDs, corticosteroids, disease-modifying antirheumatic drugs (DMARDs), anabolic steroids, antacids, antiarrhythmics, antibacterials, antibiotics, anticoagulants and thrombolytics, anticonvulsants, antidiarrheals, antiemetics, antihistamines, antihypertensives, anti-inflammatories, antineoplastics, antipyretics, antivirals, barbiturates, β-blockers, bronchodilators, cough suppressants, cytotoxics, decongestants, diuretics, expectorants, hormones, immunosuppressives, hypoglycemics, laxatives, muscle relaxants, sedatives, tranquilizers, and vitamins.

For example, the subject invention can effectively monitor concentrations of the following non-limiting list of drugs in blood: drugs for the treatment of rheumatoid arthritis or symptoms thereof, systemic lupus erythematosus or symptoms thereof, degenerative arthritis, vasculitis, inflammatory diseases, angina, coronary artery disease, peripheral vascular disease; ulcerative colitis, and Crohn's disease; anti organ rejection drugs; antiepilepsy drug; and anti-anxiety drugs.

drugs whose presence in a subject and/or concentration levels in blood can be monitored in accordance with the subject invention include, but are not limited to, the following: α-Hydroxy-Alprazolam; Acecainide (NAPA); Acetaminophen (Tylenol); Acetylmorphine; Acetylsalicylic Acid (as Salicylates); α-hydroxy-alprazolam; Alprazolam (Xanax); Amantadine (Symmetrel); Ambien (Zolpidem); Amikacin (Amikin); Amiodarone (Cordarone); Amitriptyline (Elavil) & Nortriptyline; Amobarbital (Amytal); Anafranil (Clomipramine) & Desmethylclomipramine; Ativan (Lorazepam); Aventyl (Nortriptyline); Benadryl (Dephenhydramine); Benziodiazepines; Benzoylecgonine; Benztropine (Cogentin); Bupivacaine (Marcaine); Bupropion (Wellbutrin) and Hydroxybupropion; Butabarbital (Butisol); Butalbital (Fiorinal) Carbamazepine (Tegretol); Cardizem (Diltiazem); Carisoprodol (Soma) & Meprobamate; and Celexa (Citalopram & Desmethylcitalopram).

Additional drugs whose presence and/or blood concentration levels can be monitored in accordance with the subject invention include Celontin (Methsuximide) (as desmethylmethsuximide); Centrax (Prazepam) (as Desmethyldiazepam); Chloramphenicol (Chloromycetin); Chlordiazepoxide; Chlorpromazine (Thorazine); Chlorpropamide (Diabinese); Clonazepam (Klonopin); Clorazepate (Tranxene); Clozapine; Cocaethylene; Codeine; Cogentin (Benztropine); Compazine (Prochlorperazine); Cordarone (Amiodarone); Coumadin (Warfarin); Cyclobenzaprine (Flexeril); Cyclosporine (Sandimmune); Cylert (Pemoline); Dalmane (Flurazepam) & Desalkylflurazepam; Daivocet; Darvon (Propoxyphene) & Norpropoxyphene; Demerol (Meperidine) & Normeperidine; Depakene (Valproic Acid); Depakote (Divalproex) (Measured as Valproic Acid); Desipramine (Norpramin); Desmethyldiazepam; Desyrel (Trazodone); Diazepam & Desmethyldiazepam; Diazepam (Valium) Desmethyldiazepam; Dieldrin; Digoxin (Lanoxin); Dilantin (Phenyloin); Disopyramide (Norpace); Dolophine (Methadone); Doriden (Glutethimide); Doxepin (Sinequan) and Desmethyldoxepin; Effexor (Venlafaxine); Ephedrine; Equanil (Meprobamate) Ethanol; Ethosuximide (Zarontin); Ethotoin (Peganone); Felbamate (Felbatol); Fentanyl (Innovar); Fioricet; Fipronil; Flunitrazepam (Rohypnol); Fluoxetine (Prozac) & Norfluoxetine; Fluphenazine (Prolixin); Fluvoxamine (Luvox); Gabapentin (Neurontin); Gamma-Hydroxybutyric Acid (GHB); Garamycin (Gentamicin); Gentamicin (Garaniycin); Halazepam (Paxipam); Halcion (Triazolam); Haldol (Haloperidol); Hydrocodone (Hycodan); Hydroxyzine (Vistaril); Ibuprofen (Advil, Motrin, Nuprin, Rufen); Imipramine (Tofranil) and Desipramine; Inderal (Propranolol); Keppra (Levetiracetam); Ketamine; Lamotrigine (Lamictal); Lanoxin (Digoxin); Lidocaine (Xylocalne); Lindane (Gamma-BHC); Lithium; Lopressor (Metoprolol); Lorazepam (Ativan); and Ludiomil.

The presence or blood level concentrations of the following drugs that can be monitored in accordance with the subject invention include, but are not limited to, Maprotiline; Mebaral (Mephobarbital) & Phenobarbital; Mellaril (Thioridazine) & Mesoridazine; Mephenyloin (Mesantoin); Meprobamate (Miltown, Equanil); Mesantoin (Mephenyloin); Mesoridazine (Serentil); Methadone; Methotrexate (Mexate); Methsuximide (Celontin) (as desmethsuximide); Mexiletine (Mexitil); Midazolam (Versed); Mirtazapine (Remeron); Mogadone (Nitrazepam); Molindone (Moban); Morphine; Mysoline (Primidone) & Phenobarbital; NAPA & Procainamide (Pronestyl); NAPA (N-Acetyl-Procainamide); Navane (Thiothixene); Nebcin (Tobramycin); Nefazodone (Serzone); Nembutal (Pentobarbital); Nordiazepam; Olanzapine (Zyprexa); Opiates; Orinase (Tolbutamide); Oxazepam (Serax); Oxcarbazepine (Trileptal) as 10-Hydroxyoxcarbazepine; Oxycodone (Percodan); Oxymorphone (Numorphan); Pamelor (Nortriptyline); Paroxetine (Paxil); Paxil (Paroxetine); Paxipam (Halazepam); Peganone (Ethotoin); PEMA (Phenylethylnalonamide); Pentothal (Thiopental); Perphenazine (Trilafon); Phenergan (Promethazine); Phenothiazine; Phentermine; Phenylglyoxylic Acid; Procainamide (Pronestyl) & NAPA; Promazine (Sparine); Propafenone (Rythniol); Protriptyline (Vivactyl); Pseudoephedrine; Quetiapine (Seroquel); Restoril (Temazepam); Risperdal (Risperidone) and Hydroxyrisperidone; Secobarbital (Seconal); Sertraline (Zoloft) & Desmethylsertraline; Stelazine (Trifluoperazine); Surmontil (Trimipramine); Tocainide (Tonocard); and Topamax (Topiramate).

drugs of the subject invention can be formulated to include additives (that generate detectable markers in exhaled breath) according to known methods for preparing pharmaceutically useful compositions. Formulations are described in a number of sources, which are well known and readily available to those skilled in the art. For example, Remington's Pharmaceutical Science (Martin E W [1995] Easton Pa., Mack Publishing Company, 19^{th} ed.) describes formulations that can be used in connection with the subject invention. Formulations suitable for parenteral administration include, for example, aqueous sterile injection solutions, which may contain antioxidants, buffers, bacteriostats, and solutes, which render the formulation isotonic with the blood of the intended recipient; and aqueous and nonaqueous sterile suspensions, which may include suspending agents and thickening agents.

Drug-additive formulations of the invention may be presented in unit-dose or multidose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

Administration of a drug and additive, in accordance with the subject invention, can be accomplished by any suitable method and technique presently or prospectively known to those skilled in the art. In a preferred embodiment, a drug is formulated with an additive in a patentable and easily consumed oral formulation such as a pill, lozenge, tablet, gum, beverage, etc.

According to the subject invention, a drug with an additive can be delivered from a controlled dispenser means (i.e., pill dispenser, IV bag, etc.). Upon delivery of the drug to a subject, a sensor of the invention analyzes the subject's expired gases to detect at least one target marker of the drug. Upon detection of the target marker, the subject's compliance in taking the drug is verified. In addition, where the concentration of the marker is assessed, concentration of the drug in blood can be determined for use in deriving whether the appropriate dosage amount of the drug was taken by the subject.

In one embodiment, a MAMS of the invention includes a processing system that can analyze the extent of the subject's compliance in taking the drug and can utilize the derived data based on exhaled breath analysis to provide a reminder regarding the next prescribed time to take the drug. In a related embodiment, the MAMS includes a dispenser means operatively connected to the dispenser system controller, which can dispense an appropriate dosage from the supply means to the subject based on the derived data.

Additional embodiments are also envisioned herein. Pulmonary delivery of drugs is well known, especially for conditions such as asthma and chronic obstructive pulmonary disease. In these instances, drug (i.e. corticosteroids, bronchodilators, anticholinergics, etc.) is often nebulized or aerosolized and inhaled through the mouth directly into the lungs. This allows delivery directly to the affected organ (the lungs) and reduces side effects common with enteral (oral) delivery. Metered dose inhalers (MDIs) or nebulizers are commonly used to deliver drug by this route. Recently dry powder inhalers have become increasingly popular, as they do not require the use of propellants such as CFCs. Propellants have been implicated in worsening asthma attacks, as well as depleting the ozone layer. Dry power inhalers are also being used for drugs that were previously given only by other routes, such as insulin, peptides, and hormones.

Olfactory markers can be added to these delivery systems as well. Since the devices are designed to deliver drug by the pulmonary route, the sensor array can be incorporated into the device and the subject need only exhale back through the device for documentation to occur.

Lastly, devices are available to deliver drug by the intranasal route. This route is often used for subjects with viral infections or allergic rhinitis, but is being increasing used to deliver peptides and hormones as well. Again, it would be simple to incorporate a sensor array into these devices, or the subject can exhale through the nose for detection by a marker sensing system.

Following are examples which illustrate procedures for practicing the invention. These examples should not be construed as limiting. All percentages are by weight and all solvent mixture proportions are by volume unless otherwise noted.

### EXAMPLE 1—MARKER DETECTION

To illustrate how a MAMS of the invention would operate, the following hypothetical scenario is provided, where a schizophrenic subject orally ingests an antipsychotic drug called A, which is metabolized by the liver to A1. In this example, an additive called T, is added as an excipient to the tablet of A. T is metabolized to a major metabolite, T1.

A→A1 Reaction 1

T→T1 Reaction 2

In this scenario, four candidates for use as a marker that will be measured in the breath to ideally verify that a tablet of A was ingested by the subject exist: (Option 1) the active pharmaceutic A; (Option 2) a major metabolite of the active pharmaceutic A1; (Option 3) the additive T added as an excipient to the tablet containing A; or (Option 4) the metabolite of the additive T1. These various options have distinct advantages and disadvantages. For the reasons outlined below, Option 4 is a preferred approach of the invention for selecting and preparing an additive and marker for use in a MAMS.

Option 1: The major limitation of Option 1 (detection of the active drug itself in the breath post oral ingestion of the drug) is that the MAMS will only work for that one particular active pharmaceutic, A. This is a tremendous drawback to this approach and development would only be feasible if it were carried out for widely used blockbuster-type molecules such as olanzapine with annual global sales exceeding $4 Billion. A second disadvantage of Option 1 is that the physicochemical characteristics or effective concentrations of a specific active pharmaceutic may not be suitable to allow feasible detection of this molecule in the breath. A third disadvantage of Option 1 will be a higher rate of false positives in terms of pill ingestion, particularly in those subjects with devious intentions. By detecting A rather than A1, it is possible that contamination during the process of swallowing the tablet may give a false positive indication of tablet ingestion (e.g., a devious subject could put the tablet in their mouth and then discharge it without actually ingesting the tablet). Finally, the drug may be present in breath for hours to days and therefore may not discriminate when individual doses were taken.

Option 2: The major limitation of Option 2 (detection of the major metabolites of a drug, A1 in the breath post oral ingestion of the drug) is the same as that of Option 1. However, Option 2 has one significant advantage over Option 1. In contrast, if A1 were promptly detected in the breath, and these entities are created by the action of a specific enzyme in the liver, it guarantees that the subject put the pill in his/her mouth and that it traveled down the esophagus into the gastrointestinal tract (e.g., stomach, small intestine), where it was absorbed into the blood and sent to the liver for metabolism. Nevertheless, Option 2 still limits the system to detecting ingestion of a specific active drug. Furthermore, like the case in Option 1, the metabolites of the active pharmaceutic, A1, may not have the appropriate physicochemical characteristics and/or concentration profile at doses of A for feasible detection in breath. On the other hand, in select circumstances it may be feasible to successfully measure A1 in the breath using a MAMS of the invention, particularly if it has a half life that is short enough relative to the half life of A and if it contains distinctive chemical moieties that can be readily measured with COTS sensing technologies (e.g., amides, sulfur and/or fluorine-containing molecules).

Option 3: The major advantage of Option 3 (detection of T that is placed as an excipient of a pill containing A) is that it not only allows the selection of a chemical additive that possesses the attributes of the ideal marker (as described above), but it can be utilized to verify oral ingestion of any active pharmaceutic as opposed to one particular drug (limitations of Options 1 and 2). However, by utilizing T, rather than a metabolite of T (Option 4), it cannot guarantee that the tablet was ingested (i.e., surface contamination in the mouth of T could give a false positive on a MAMS) if it is simply incorporated as an excipient into the tablet (surface or incorporation into matrix). This drawback can be significantly mitigated or even eliminated by pill design factors such as how it constructed (e.g., a pill consisting of a capsule with an outer pH-sensitive layer; an acidic environment like that in the stomach dissolves the coating and releases its contents only when it is exposed to the stomach).

Option 4: The preferred approach of the four options for a MAMS of the invention is Option 4 (detection of T1) where A and T co-exist in the same pill. Option 4 has 3 major advantages: (1) allows the selection of a chemical additive that possesses the attributes of the ideal marker, (2) can be utilized to verify oral ingestion of any active pharmaceutic, and (3) can guarantee that the active pharmaceutic was ingested, entered the blood compartment, traveled to its biological target sites and via its mechanism(s) underlying efficacy exerted its action. For example, if an enzyme, which is located in the liver, converts T to T1, then detection of T1 in the breath definitively confirms pill ingestion of active drug in the subject who actually put the tablet in his/her mouth. As contemplated herein, the enzyme system used to generate T1 does not have to be confined to the liver but could include other extrahepatic enzyme systems such as blood esterases, etc.

### EXAMPLE 2—DEVELOPMENT OF A DRUG ADHERENCE MONITORING SYSTEM (MAMS)

In this prototypical example novel chemistry will be employed to create a series of non-toxic (at concentrations required for MAMS application in accordance with the present invention), non-endogenous, highly distinctive compounds (e.g., fluoroalcohols, fluoroaldehydes) to be liberated in vivo and appear in the breath for an optimal period time for MAMS and be easily detected by real time accurate point of care COTS devices that are currently marketed for other applications. The design and construction of a preferred MAMS of the invention is dependent upon two critical components: (a) development of novel chemistry to generate the marker, and (b) development of COTS sensing technology to measure the marker. In some embodiments of a MAMS of the invention, the system can include any one or combination of the following elements: (1) alveolar gas sampler, (2) communication link to notify user of marker detection, etc.

### Preparation of Additive

According to the present invention, an additive to be combined with a drug includes an ester-based compound, which is comprised of R groups (see FIG. 1, Table 1 for examples of R groups that can be used) and R′ groups (see Table 2) on the additive structure. The R and/or R′ groups (including but not limited to alkyl groups in the area of the carbonyl moiety) preferably establishes the susceptibility of the ester bond of the additive to hydrolysis. By varying the degree of steric hindrance and/or electronic interaction between the ester and the esterases, the nature and size of R and/or R′ can markedly alter the rate of ester hydrolysis and hence the rate of generation of the detectable alcohol (R′—OH) (also referred to herein as the marker).

FIG. 1 illustrates hydrolysis of an ester to a carboxylic acid and alcohol. According to the subject invention, the alcohol is the drug marker detectable in exhaled breath, where the R and R′ group can be varied in accordance with the groups described in Table 1. The R and R′ groups selected can be used to regulate the rate of hydrolytic conversion in the subject of the ester to the alcohol.

In one embodiment, the R′—OH marker is a fluoroalcohol. Each type of fluoroalcohol (R′—OH), which will serve as the marker in this example, has unique physicochemical characteristics (e.g., vapor pressure, boiling point, melting point, flash point, lipophilicity, etc.) and metabolic half lives in humans. In addition, volatile fluorine molecular entities, unlike those of others such as sulfur compounds or amines that can be generated endogenously in humans, are not naturally found in humans. Thus, fluorinated alcohols will serve as a highly distinctive marker of pill ingestion, which can be easily detected using inexpensive, real time, COTS sensor devices.

According to the present invention, the tertiary fluoroalcohols (derived from compounds R′1, R′2 and R′3) should be particularly resistant to metabolism. Given the relative volatility and stability of fluoroalcohols in blood, a significant fraction of the fluoroalcohol markers derived from ester-based additives of the invention should be excreted from the body via the lungs in the breath.

As outlined above, one aspect of the invention includes methods of engineering a marker that will appear and continue to be present in the breath for an appropriate period of time. In this example, a total of 10 R groups and a total of 4 R′ groups are provided in Tables 1 and 2, respectively. Thus, a total of 40 new molecular entities could be readily synthesized with this approach for use as additives to be added to drugs, contingent upon compound stability and ease of compound synthesis. The R and R′ groups listed in the Tables 1 and 2 are not all inclusive and can be easily expanded to include other chemical entities.

According to the present invention, the duration that the marker, which is a fluoroalcohol in this example, can persist in the breath is a function of two different factors: (1) the rate of liberation of the fluoroalcohol from the hydrolysis of the ester (a function of R and R′ group substitution), and (2) the intrinsic properties of the fluoroalcohol (e.g., a function of physicochemical properties such as vapor pressure at physiological temperature and pharmacokinetic features such as metabolic half life, clearance and volume of distribution). By using use the combination of 40 R and R′ ester-based additive entities, the present invention provides a novel and advantageous method for identifying an ideal fluoroalcohol for use as a marker in a MAMS.

### Sensor Technology—COTS

By utilizing ester-based additive chemistry to generate fluoroalcohols as markers detectable in exhaled breath, a number of COTS sensor devices, each with essentially ideal performance characteristics for use in a MAMS of the invention, can be readily employed in this invention. These devices have been used to detect leaks of Freons, hydrofluorocarbons and chlorofluorocarbons (CFCs). Preferred types of COTS for use in the present example include, but are not limited to: Negative Corona Leak Detector (e.g., TIF TIFXP-1A); Negative Ion Capture (e.g., Ion Science SF6 Leak Check P1 and Gas Check P1); Heated Sensor/Ceramic Semiconductor (e.g., Yellow Jacket Accuprobe; Ion Science GasCheck R2 pc); and Infrared Absorption (e.g., PSICORP GasScan Miniature Diode Laser-based Ambient Gas Sensor; INFICON D-Tek IR Leak Detector; BACHARACH H25-IR Leak Detector).

The sensors listed above are ideal for a MAMS of the invention because they are compact/portable, highly reliable, operate real time, sensitive/specific, easy to use, operate with minor interferences (e.g., humidity), and are inexpensive. Likewise, these types of sensors can be very easily integrated into cell phones (or computers) containing an alveolar gas collection system (not likely to be needed) and interfaced to communication links, which can be used to link drug adherence to outside monitors (e.g., family, central agency, hospital, doctor's offices).

TABLE 1
Different R group substitutions on the ester

| Number | R Name | R Structure |
|---|---|---|
| R1 | Methyl | —CH₃ |
| R2 | Ethyl | —C₂H₅ |
| R3 | Isopropyl | —CH(CH₃)₂ |
| R4 | Isobutyl | —CH₂CH(CH₃)₂ |
| R5 | sec-Butyl | CH(CH₃)CH₂CH₃ |
| R6 | Neopentyl | —CH₂C(CH₃)₃ |
| R7 | Cyclohexyl | —C₆H₁₁ |
| R8 | (R)-Menthyl | 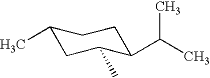 |
| R9 | (R)-endo-Bornyl | 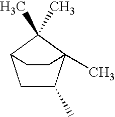 |
| R10 | Adamantanemethyl | 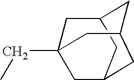 |

TABLE 2
Fluoroalcohols that can potentially serve as the exhaled drug ingestion marker
(EDIM) in breath following hydrolysis of the ester taggant

| | | | CAS Code | |
|---|---|---|---|---|
| | | R′—OH | Molecular Formula | Physical |
| Code | R′ | Name of Fluoroalcohol | Molecular Weight | Properties |
| R′1 | (CF₃)₂CH₃— | 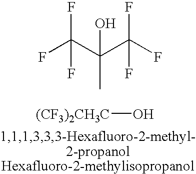 | CAS: 1515-14-6MF: C₄H₄F₆OMW: 182.07 | BP: 60° C.MP: N/A ° C.sg: 1.484 g/mlFlash P: 113° C. |
| R′2 | CF₃(CH₃)₂C— | 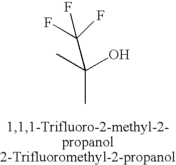 | CAS: 507-52-8MF: C₄H₇F₃OMW: 128.09 | BP: 80° C.MP: N/A ° C.sg: N/A g/mlFlash P: N/A |
| R′3 | (CF₃)₃C— | 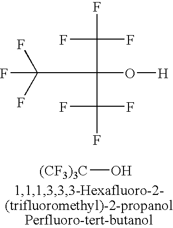 | CAS: 2378-02-1MF: C₄HF₉OMW: 236.04 | BP: 45° C.MP: N/A ° C.sg: 1.69 g/mlFlash P: N/AVP: 268 torr at 20° C.Solubility:39,200 mg/Lwater at 25° C. |
| R′4 | 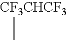 | 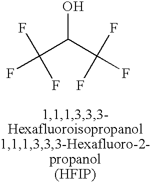 | CAS: 920-66-1MF: C₃H₂F₆OMW: 168.04HFIP is a majormetabolite of thevolatile anesthetic,sevoflurane | BP: 58.2° C.MP: −3.3° C.sg: 1.596 g/mlFlash P: >100° C.VP: 102 torr at20° C.; 159 torr at25° C.Solubility:7770 mg/L water at25° C.;pKa = 9.3 at25° C.LD₅₀/LC₅₀:Inhalation rat:LC₅₀ 1974 ppm/4H; Oralrat: LD501040–1500 mg/kg |

TABLE 3
Physicochemical Properties of Relevant Aliphatic Alcohols

| R′—OH | CAS Code | |
|---|---|---|
| Name of | Molecular Formula | |
| Aliphatic Alcohol | Molecular Weight | Physical Properties |
| 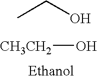 | CAS: 64-17-5MF: C₂H₆OMW: 46.07 | BP: 78° C.MP: −114.1° C.sg: 0.79 g/mlFlash Point: 16.6° C.VP: 59.3 torr at 20° C.Water solubiltiy: miscibleLD₅₀/LC₅₀: Inhalation rat: LC₅₀ 2000 ppm/10H; Oral rat: LD₅₀ 7060–9000 mg/kg |
| 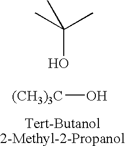 | CAS: 75-65-0MF: C₄H₁₀OMW: 74.12 | BP: 83° C.MP: 25° C.sg: 0.786 g/mlFlash Point: 11° C.VP: 31 torr @ 20° C.LD₅₀/LC₅₀: Inhalation rat: LC₅₀>10,000 ppm/4H; Oral rat: LD₅₀2743–3500 mg/kg |

### EXAMPLE 3—ADDITIVE SELECTION AND SYNTHESIS

To further demonstrate how chemistry can be easily modified to generate a marker in exhaled breath, in this illustrative example, the additive used to generate the marker is a phosphate compound, which is hydrolytically degraded by alkaline hydrolysis through the enzyme alkaline phosphatase (FIG. 2). The resulting products are an alcohol, a ketone, and a phosphate. Similar to Example 2, the rate of hydrolysis by alkaline phosphatase can be regulated by the degree of steric/electronic hindrance put on the bond via substitutions at R′, R″ and/or R′″ positions. Possible R′, R″ and/or R′″ groups are shown, but not limited to those depicted in Table 4. According to the present example, if the R″ and R′″ groups each contain a simple H atom, then the ketone generated in FIG. 2 is the aldehyde, formaldehyde (HCOH). In this particular example, the alcohol (via the R′ group) generated will be a fluoroalcohol. Like Example 2, possible fluoroalcohols generated as the marker are shown but not limited to those listed in Table 2.

In addition to the two enzyme systems described in the examples presented herein (i.e., esterase, alkaline phosphatase), a number of other enzyme systems (e.g., various fractions of CYP/P₄₅₀ system) could also be readily used to generate the marker from an additive. In addition to using fluoroalcohols as markers as outlined in the examples, a number of other molecular entities including, but not limited to sulfur containing compounds or amides could be used as markers of the invention. Many of these compounds are natural compounds, either produced within humans or frequently ingested as food, which could be used directly as the additive or could be generated in vivo to provide a detectable marker using the enzyme approaches (e.g., esterase, alkaline phosphatase, CYP) outlined above.

Portable real time COTS sensor devices can be used to sensitively and specifically measure markers other than fluoroalcohols (e.g., sulfur, amides). With fluorinated compounds as the marker, baseline breath measurements are likely to not be necessary. Use of a marker(s) that appears naturally in the body may (or may not) require baseline measurements in order to detect drug ingestion.

Monitoring of subject adherence to multiple drugs is feasible in this invention, if a COTS device can be coupled to a particular marker, which in turn is linked to a specific drug. For example, to monitor adherence in a subject prescribed three different drugs, an additive unique to each drug would each generate a marker that would be detected by different sensing technologies in a COTS sensor device (e.g., fluoroalcohol for drug A; natural sulfur compound for drug B; and natural amine for drug C).

TABLE 4
Different R′, R″ and/or R′′′ group substitutions to vary
the rate of hydrolysis via alkaline phosphatase

| R′, R″ and/or | |
|---|---|
| R′′′ Name | Structure |
| Hydrogen | —H |
| Methyl | —CH₃ |
| Ethyl | —C₂H₅ |
| Isopropyl | —CH(CH₃)₂ |
| Isobutyl | —CH₂CH(CH₃)₂ |
| sec-Butyl | CH(CH₃)CH₂CH₃ |
| Neopentyl | —CH₂C(CH₃)₃ |
| Cyclohexyl | —C₆H₁₁ |
| (R)-Menthyl | 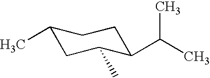 |
| (R)-endo-Bornyl | 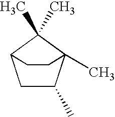 |
| Adamantanemethyl | 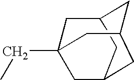 |

### EXAMPLE 4—SYNTHESIS OF CYP 2D6 SUBSTRATE ADDITIVE

As noted above, in addition to the esterase and alkaline phosphatase enzyme systems, a number of other enzyme systems (e.g., various fractions of CYP/P₄₅₀ system) could be readily used to generate a marker from an additive. In addition to using fluoroalcohols as markers as outlined in the examples above, a number of other molecular entities including, but not limited to fluorous carbonyl compounds that are volatile (to the same extent if not more so than fluoroalcohols), can be used as markers in accordance with the present invention. Many such fluorous carbonyl compounds could be generated in vivo (and excreted in exhaled breath for detection) from an additive that is metabolized via various isoforms of the CYP/P₄₅₀ enzyme system. An example of such an additive and resultant fluorous carbonyl compound/marker are provided below.

### Materials and Methods

### General Chemical Methods

All reagents were purchased from Sigma (St. Louis, Mo.). All solvents used were reagent grade and obtained from Fisher Scientific (Atlanta, Ga.). Column chromatography was carried out on Fisher silica gel (230-400 mesh). Thin-layer chromatography (TLC) analyses were performed on Fisher silica gel 60-F₂₅₄ plates and visualized using UV light (254 nm). ¹H NMR (300 MHz) spectra were recorded on a Varian Unity 300 spectrometer. Chemical shifts are given in parts per million (ppm). Only diagnostic peaks are reported. APCI mass spectra were obtained using a Thermo Finnigan (San Jose, Calif.) LCQ mass spectrometer. Elemental (combustion) analyses were performed by Atlantic Microlab, Inc. (Norcross, Ga.).

### Synthesis of O-trifluoroethyl dextrorphan

3-(O-Desmethyl)Dextromethorphan (2): Dextromethorphan hydrobromide 1 (10 g, 28.4 mmol) was dissolved in 48% aqueous hydrobromic acid (50 ml). The solution was heated to reflux for 18 hours. The mixture was poured on crushed ice, and treated with K₂CO₃ until pH=10. The mixture was extracted with chloroform (3×100 mL).

The combined organic extracts were washed with brine, dried over Na₂SO₄, filtered, and the solvent was removed at reduced pressure to give a solid (6 g, 81%). Analysis using thin layer chromatography (TLC) showed very little of the starting material remained, R_{f}=0.2 (95:5 CH₂Cl₂/MeOH), where the material was used without further purification. ¹H NMR (CDCl₃): δ 6.97 (d, J=8.1 Hz, 1H, C1-H), 6.72 (d, J=2.4 Hz, 1H, C4-H), 6.61 (dd, J=2.7, 8.1 Hz, 1H, C2-H), 2.40 (s, 3H, N—CH₃); MS (APCI): m/z 258 [M+1]⁺.

N,O-bis(vinyloxycarbonyl)morphinan (3): 2 (6 g, 23.4 mmol) and Proton Sponge (1,8-bis(dimethylamino)naphthalene, 6 g, 28.2 mmol) were dissolved in 1,2-dichloroethane (50 mL) at 60° C. under N₂. After adding vinyl chloroformate (6 g, 53 mmol), the solution was heated at reflux overnight. TLC revealed no starting material remaining. The mixture was filtered and concentrated, and the residue was purified by column chromatography eluting with CH₂Cl₂. Combination of the desired fractions followed by solvent removal gave a yellow oil (5 g, 56%), R_{f}=0.67 (CH₂Cl₂). ¹H NMR (CDCl₃): δ 7.28 (d, J=8.1 Hz, 1H, C1-H), 7.20 (m, 2H, H₂C═CHO—COR, R═N and O), 7.11 (s, 1H, C4-H), 7.01 (dd, J=2.4, 8.1 Hz, 1H, C2-H), 5.04 (dd, J=2.1, 13.8 Hz, 1H, trans-H₂C═CHO—COO), 4.77 (d, J=14.4 Hz, 1H, trans-H₂C═CHO—CON), 4.68 (dd, J=2.1, 6.3 Hz, 1H, cis-H₂C═CHO—COO), 4.45 (d, J=6.9 Hz, 1H, cis-H₂C═CHO—CON); MS (APCI): m/z 384 [M+1]⁺.

3-Hydroxy-N-vinyloxycarbonyl-morphinan (4): 3 (3.27 g, 8.5 mmol) was dissolved in dioxane (36 mL) and water (12 mL) containing 408 mg (10.2 mmol) of NaOH. The solution was heated at 60° C. for 3 hours. TLC revealed no starting material present. The mixture was cooled to room temperature, poured into brine, and extracted with ether (3×50 mL). The combined ether extracts were dried over Na₂SO₄, filtered, and solvent was evaporated in vacuo to leave a residue, which was purified by column chromatography eluting with 10-50% EtOAc in Hexane to yield an oil (2.5 g, 94%), R_{f}=0.29 (CH₂Cl₂). ¹H NMR (CDCl₃): δ 7.18 (m, 1H, NCOOCH═CH₂), 6.93 (d, J=6.6 Hz, 1H, C1-H), 6.76 (d, J=1.8 Hz, 1H, C4-H), 6.64 (dd, J=2.1, 6.3 Hz, 1H, C2-H), 4.69 (dd, J=0.9, 10.5 Hz, 1H, trans-H₂C═CHO—CON), 4.37 (dd, J=0.9, 4.8 Hz, 1H, cis-H₂C═CHO—CON); MS (APCI): m/z 314 [M+1]⁺.

2,2,2-Trifluoroethyl p-toluenesulfonate (5): p-toluenesulfonyl chloride (4.5 g, 24 mmol) dissolved in CH₂Cl₂ (10 mL) was added dropwise under N₂ to a solution of 2,2,2-trifluoroethanol (1.6 g, 16 mmol) in 10 mL CH₂Cl₂, followed by 4.5 mL TEA at 0° C. After completion of adding TEA, reaction was stirred at room temperature for 16 hours. The mixture was concentrated, and the residue was dissolved in EtOAc (150 mL), washed with NaHCO₃ (100 mL), 0.5 M Citric acid (100 mL), distilled water (100 mL), dried over Na₂SO₄. Solvent was evaporated in vacuo to leave a residue, which was purified by column chromatography eluting with 10% EtOAc in Hexane to yield product 5 (3.2 g, 80%), R_{f}=0.33 (4:1 Hexane/CH₂Cl₂). ¹H NMR (CDCl₃): δ 7.75 (d, J=6.9 Hz, 2H, aromatic H), 7.33 (d, J=6.9 Hz, 2H, aromatic H), 4.32 (q, J=7.8 Hz, 2H, CH₂CF₃), 2.38 (s, 3H, CH₃).

3-(2,2,2-Trifluoroethyl)-N-vinyloxycarbonyl-morphinan (6): To a rapidly stirred solution of 4 (2 g, 6.4 mmol) in dry DMF (25 mL) was added NaH (60% oil dispersion, 400 mg, 10 mmol) under N₂. After stirring for 1 hour, 5 (2.3 g, 9.1 mmol) in 10 mL of DMF was added dropwise. The mixture was stirred at room temperature overnight and then poured into 100 mL brine, extracted with ether (3×40 mL). The combined organic extracts were dried over Na₂SO₄, filtered and concentrated in vacuo. The residue was purified by column chromatography eluting with CH₂Cl₂/MeOH to yield an oil (1 g, 40%), R_{f}=0.22 (4:1:0.1 Hexane/CH₂Cl₂/MeOH). ¹H NMR (CDCl₃): δ 7.18 (m, 1H, NCOOCH═CH₂), 6.93 (d, J=6.6 Hz, 1H, C1-H), 6.76 (d, J=1.8 Hz, 1H, C4-H), 6.64 (dd, J=2.1, 6.3 Hz, 1H, C2-H), 4.69 (dd, J=0.9, 10.5 Hz, 1H, trans-NCOOCH═CH₂), 4.37 (dd, J=0.9, 4.8 Hz, 1H, cis-NCOOCH═CH₂): m/z 396 [M+1]⁺.

O-trifluoroethyldextrorphan (7): 6 (730 mg, 1.84 mmol) was dissolved in dry THF (25 mL) and stirred in ice bath for 30 minutes under N₂. LiAlH₄ (300 mg, 7.9 mmol) was added in small portions with rapid stirring at 0° C. The mixture was stirred overnight at room temperature. To the mixture was added 0.3 mL water and 0.3 mL of 15% aqueous NaOH. The mixture was poured into 75 mL of water, extracted with ether (4×30 mL). The combined organic extracts were dried over Na₂SO₄, filtered and concentrated in vacuo. The residue was purified by column chromatography eluting with 5-20% MeOH in CH₂Cl₂ to yield an oil (360 mg, 58%), R_{f}=0.2 (95:5 CH₂Cl₂/MeOH). ¹H NMR (CDCl₃): δ 7.05 (d, J=8.1 Hz, 1H, C1-H), 6.85 (d, J=2.7 Hz, 1H, C4-H), 6.70 (dd, J=2.7, 8.1 Hz, 1H, C2-H), 4.32 (q, J=8.1 Hz, 2H, CF₃CH₂), 2.39 (s, 3H, NCH₃); MS (APCI): m/z 340 [M+1]⁺. The oil (60 mg) was dissolved in diethyl ether (1 mL) and treated with 48% aqueous HBr (200 μL), then evaporated in vacuo and dried to get the white solid hydrobromide. Analysis (C₁₉H₂₄F₃NO.1.5HBr.0.6H₂O): C, H, N.

### CYP 2D6 Inhibition Assays

CYP 2D6 inhibition assays were performed by Novascreen (Hanover, Md.). In brief, after 100 μL solution containing the compounds (dextromethorphan or trifluoroethyldextrorphan, concentration ranging from 10⁻¹⁰ M to 10⁻⁵M) and mixed 50 μL cofactor solution were preincubated in 100 mM potassium phosphate buffer (pH=7.4) for 10 min at 37° C., 50 μL of freshly mixed solution of human recombinant CYP2D6 (1.5 pmole) and AMMC (1.5 μM) was added to each well. The final cofactor concentrations were 1.3 mM NADP⁺, 3.3 mM glucose-6-phosphate, 0.4 U/ml glucose-6-phosphate dehydrogenase. Samples were incubated for 45 minutes and the reaction was stopped with 75 μL Tris/acetonitrile stop solution. Quinidine was used as the reference compound. The AMMC metabolite, AMHC, was measured using a fluorescent plate scanner at an excitation wavelength of 390 nm and emission wavelength of 460 nm. As shown in FIG. 5, the potency to inhibit AMMC metabolism was similar for dextromethorphan and trifluoroethyldextrorphan.

### Human Liver Microsomes Assays

Pooled human liver microsomes and NADPH regenerating system were obtained from Gentest (Woburn, Mass.) and stored at −80° C. LpDNPH S10 cartridge was from Supelco (Bellefonte, Pa.). All solvents were HPLC grade and were purchased from Fisher Scientific (Atlanta, Ga.).

Microsomal incubations were conducted in 1.5 mL polypropylene vials containing 2 mg/mL human liver microsome, 5.2 mM NADP⁺, 13.2 mM glucose-6-phosphate, 1.6 U/ml glucose-6-phosphate dehydrogenase, 6.6 mM magnesium chloride and 1 mM trifluoroethyldextrorphan in 100 mM potassium phosphate buffer (pH 7.5). The total volume was 1.5 mL.

LpDNPH S10 cartridges were employed to collect trifluoroacetaldehyde produced from incubation mixture for 1.5 hours through suction generated by a 5 mL syringe. The incubations were performed in triplicate. After 1.5 hours incubation, cartridges were washed with 3 mL acetonitrile and 20 μL [¹⁵N₄]trifluoroacetaldehyde 2,4-dinitrophenylhydrazone (internal standard, 50 μg/mL) was added. Acetonitrile was removed under nitrogen stream, the residue was dissolved in 20 μL dichloromethane and 2 μL was used for GC/MS analysis.

### Gas Chromatography-Mass Spectrometry

A Thermo Finnigan Polaris Q Mass Spectrometer (MS) system (consists of a Polaris Q MS, a TRACE GC, and the Xcalibur Data System) was used. GC conditions were as follows: Column, Rtx®-5MS silica capillary column (30 m×0.25 mm i.d., 0.25 μm); helium carrier gas at a flow rate of 1 mL/min; injector temperature 220° C.; column head pressure 7.8 psi. The initial oven temperature was 40° C. for 1 min, increased to 300° C. at 25° C./min and maintained at 300° C. for 3 min. All injections of samples were carried out using the splitless mode. MS conditions were: ion source temperature, 200° C.; interface temperature, 300° C.; ionizing voltage, 70 eV. Negative chemical ionization (NCI) mode was used for identification and quantitation of trifluoroacetaldehyde 2,4-dinitrophenylhydrazone. Methane was used as a reagent gas.

[¹⁵N₄]trifluoroacetaldehyde 2,4-dinitrophenylhydrazone was used as an internal standard for the determination of trifluoroacetaldehyde as its DNPH derivative. Quantitation was performed by selected ion monitoring (SIM) of most intensive fragment ions trifluoroacetaldehyde 2,4-dinitrophenylhydrazone (m/z 182) against the internal standard [¹⁵N₄]trifluoroacetaldehyde 2,4-dinitrophenylhydrazone (m/z 185).

### Results

### Chemistry

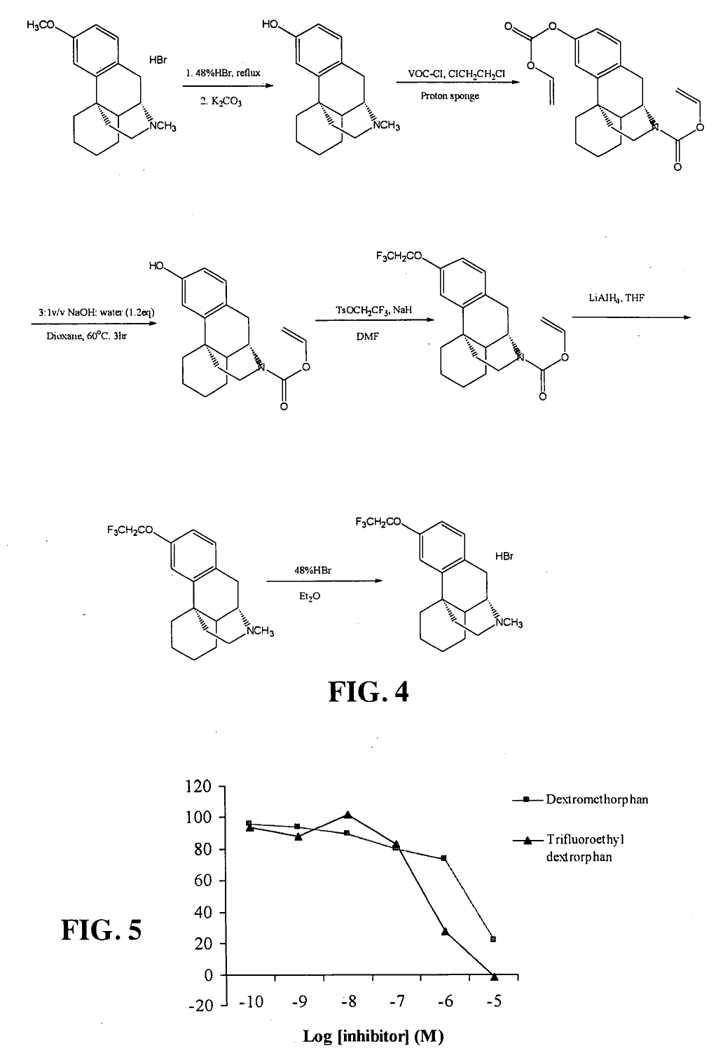

Synthesis of O-trifluoroethyldextrophan involved a five-step procedure (see, for example, methods of Senderoff S G, Landvatter S W and Heys J R, 2000 and Olofson R A and Schnur R C, 1977, “Value of the vinyloxycarbonyl unit in hydroxyl protection: application to the synthesis of nalorphine” Tetrahed. Lett. 18: 1571-1574). First, dextromethorphan hydrobromide was O-demethylated by treatment with 48% aqueous hydrobromic acid at reflux and the resulting O-desmethyl dextromethorphan hydrobromide was converted to the free base. Next, this free base was treated with 2.25 eq of vinylchlroformate and Proton Sponge™ in 1, 2 dichloroethane to give bis-vinyloxycarbonyl derivative. Then, bis-vinyloxycarbonylmorphinan was selectively O-deprotected by hydrolysis with dioxane/aqueous sodium hydroxide (3:1) at 60° C. In the fourth step, the resulting N-vinyloxycarbonyl phenol was treated with 2,2,2-trifluoroethyl p-toluenesulfonate (made by tosylation of 2,2,2-trifluoroethanol using p-toluenesulfonyl chloride) and sodium hydride in N,N-dimethylformamide to give 3-trifluoroethoxy N-vinyloxycarbonylmorphinan (Sonesson C, Lin C H, Hansson L, Waters N, Svensson K, Carlsson A, Smith M W and Wikstrom H., 1994). Last, this trifluoroethyl derivative was reduced by lithium aluminum hydride in tetrahydrofuran to give O-trifluoroethyl dextrorphan free base and the final compound was obtained by treating this free base with 48% hydrobromic acid. The synthesis is shown in FIG. 4.

### Determination of Trifluoroacetaldehyde

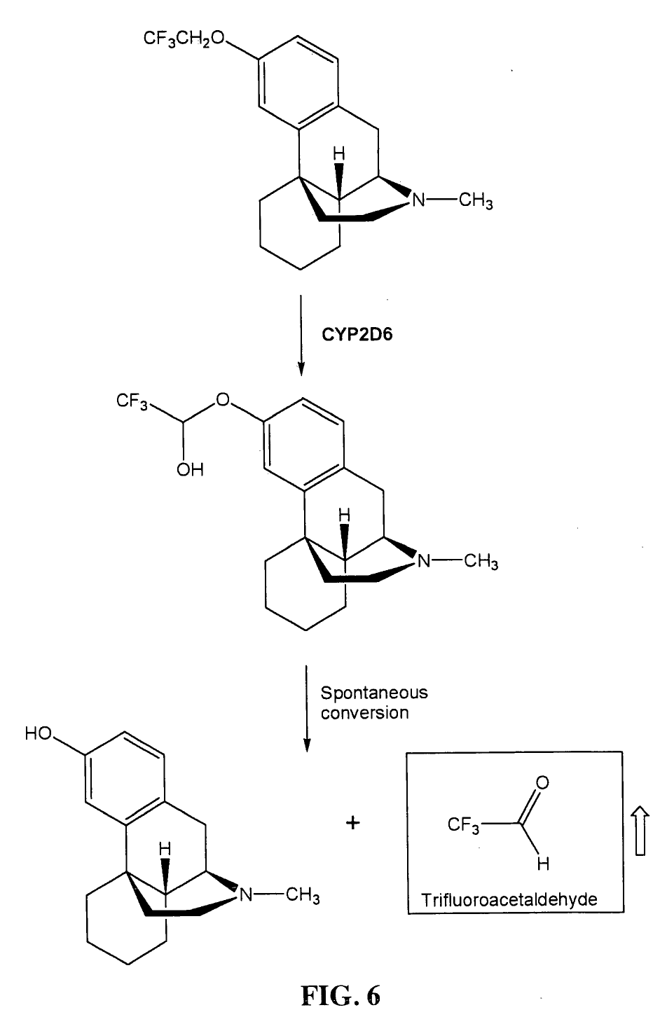

According to the subject example, trifluoroacetaldehyde (a detectable marker) should be produced after detrifluoroethylation of trifluoroethyl dextrorphan (an additive). Specifically, as illustrated in FIG. 6, metabolism of the additive, trifluoroethyldextrorphan, via a CYP enzyme system (such CYP 2D6) yields a volatile trifluoroacetaldehyde marker that is readily detected in exhaled breath as an indication of subject compliance in taking the drug (which was administered concurrently with the additive). In order to detect its formation, trifluoroacetaldehyde was collected during 90 minutes of microsomal incubation. Due to the high volatility and reactivity of the aldehyde, it is usually derivatized first before determination to fix its concentration at a given time.

DNPH is the most commonly used reagent for derivatization (Kolliker S, Oehme M and Dye C, 1998, “Structure Elucidation of 2,4-Dinitrophenylhydrazone Derivatives of Carbonyl Compounds in Ambient Air by HPLC/MS and Multiple MS/MS Using Atmospheric Chemical Ionization in the Negative Ion Mode” Anal Chem 70: 1979-1985). Trifluoroacetaldehyde released into the headspace was derivatized to its DNPH adduct using solid-phase capture cartridge before analysis by GC/(NCI)MS. Analysis is often performed by GC equipped with flame ionization (Priego-Lopez E, Luque de Casto M D, 2002, “Pervaporation-gas chromatography coupling for slurry samples. Determination of acetaldehyde and acetone in food” J Chromatogr A 2002, 976:399-407), electron capture (Ohata H, Otsuka M and Ohmori S, 1997, “Determination of acetaldehyde in biological samples by gas chromatography with electron-capture detection” J Chromatogr B Biomed Sci Appl. 693: 297-305; Tomita M, Okuyama T, Hatta Y and Kawai S, 1990, “Determination of free malonaldehyde by gas chromatography with an electron-capture detector” J. Chromatogr B Biomed. Appl. 526: 174-179) or mass spectrometric detection (Park H M, Eo Y W, Cha K S, Kim Y M and Lee K B, 1998, “Determination of free acetaldehyde in total blood for investigating the effect of aspartate on metabolism of alcohol in mice” J Chromatogr B Biomed Sci Appl. 719: 217-221).

Liquid chromatography (Lucas D, Menez J F, Berthou F, Pennec Y and Floch H H, 1986, “Determination of free acetaldehyde in blood as the dinitrophenylhydrazone derivative by high-performance liquid chromatography” J Chromatogr 382: 57-66; Shara M A, Dickson P H, Bagchi D and Stohs S J, 1992, “Excretion of formaldehyde, malondialdehyde, acetaldehyde and acetone in the urine of rats in response to 2,3,7,8-tetrachlorodibenzo-p-dioxin, paraquat, endrin and carbon tetrachloride” J. Chromatogr B Biomed. Appl. 576:221-233), liquid chromatography-mass spectrometry (LC/MS; Kolliker S, Oehme M and Dye C, 1998, “Structure Elucidation of 2,4-Dinitrophenylhydrazone Derivatives of Carbonyl Compounds in Ambient Air by HPLC/MS and Multiple MS/MS Using Atmospheric Chemical Ionization in the Negative Ion Mode” Anal Chem 70: 1979-1985; Kempter C, Zurek G and Karst U, 1999, “Determination of carbonyls using liquid chromatography-mass spectrometry with atmospheric pressure chemical ionization” J Environ Monit 1: 307-311; Zurek G and Karst U, 1999, “Liquid chromatography-mass spectrometry method for the determination of aldehydes derivatized by the Hantzsch reaction” J. Chromatogr A 864: 191-197) and liquid chromatography-tandem mass spectrometry (LC/MS/MS) (Nagy K, Pollreisz F, Takats Z and Vekey K, 2004, “Atmospheric pressure chemical ionization mass spectrometry of aldehydes in biological matrices” Rapid. Commun. Mass. Spectrom. 18: 2473-2478) have also been used.

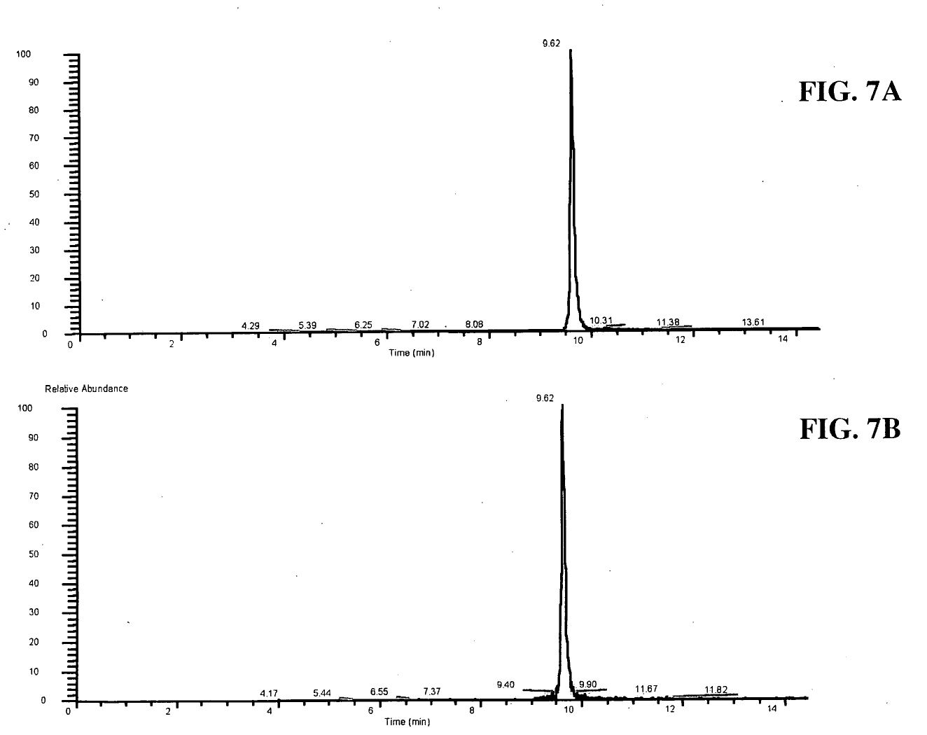

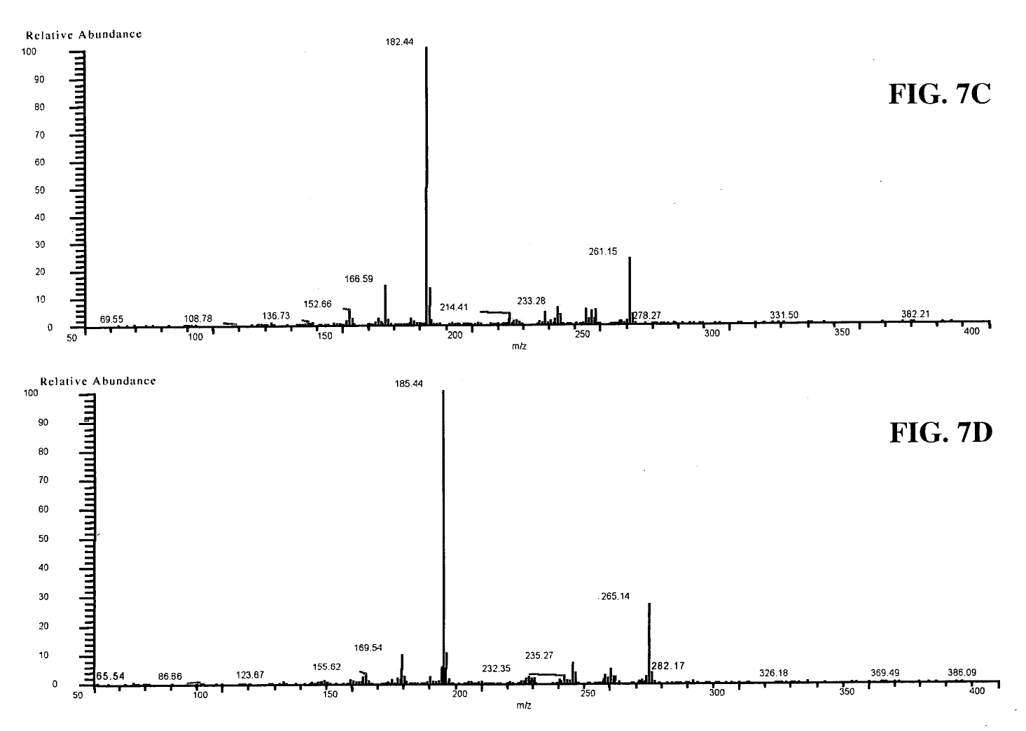

To confirm the structure of trifluoroethylaldehyde 2,4-dinitrophenylhydrazone, the standard compound was synthesized by treating trifluoroacetaldehyde ethylhemiacetal with DNPH in toluene using p-toluenesulfonic acid as a catalyst and heating the solution under reflux for 4 hours (Abouabdellah A, Begue J P, Bonnet-Delpon D and Nga T T T, 1997, “Diastereoselective synthesis of the nonracemic methyl syn-(3-fluoroalkyl)isoserinates” J. Org. Chem. 62: 8826-8833; Guanti G, Banfi L, Narisano E, Scolastico C and Bosone E, 1985, “Monobactams: stereoselective synthesis of trans-3-amino- and 3-acylamino-4-trifluoromethyl-2-azetidinones” Synthesis 6/7: 609-611). A ¹⁵N-labeled trifluoroacetaldehyde 2,4-dinitrophenylhydrazone was also synthesized as an MS internal standard using labeled DNPH (Prokai and Forster, US Patent Application, Ser. No. 60/614,951). The analyte and internal standard have the same retention time, but show 4-u difference in m/z of molecular ions. Since fragmentation occurs between the two N, m/z of the two intense fragment ions differs by 3 u. FIGS. 7A-D show the chromatogram and mass spectra of these two compounds. The fragment ions m/z 182 and 185 were monitored for quantitation. The amount of trifluoroacetaldehyde 2,4-dinitrophenylhydrazone captured by the LpDNPH S10 cartridge was calculated by multiplying the ratio of the analyte to internal standard peak areas of the SIM chromatograms with the known quantity (1 μg) of the internal standard added. This assay method indicated that 120 ng trifluoroacetaldehyde was captured from the headspace of the microsomal incubation mixture used to confirm the metabolic generation of a potential fluorous exhaled breath marker. Accordingly, trifluoroethyldextrorphan would be an excellent additive to be combined with a drug.

According to the subject invention, other analogs of trifluoroethyldextrorphan can be used as an additive. For example, trifluoropropyl dextrorphan and trifluorobutyl dextrorphan can be used as additives, in which trifluorinated aldehydes are produced after detrifluoroethylation in a subject. These analogs are equally as effective, if not more so, as trifluoroethyldextrorphan in producing detectable markers in exhaled breath samples.

### EXAMPLE 5—SELECTION OF SENSORS

The following are examples of various sensor technologies that may be utilized in practicing the method of the present invention:

### Microgravimetric Sensors

Microgravimentric sensors are based on the preparation of polymeric- or biomolecule-based sorbents that are selectively predetermined for a particular substance, or group of structural analogs. A direct measurement of mass changes induced by binding of a sorbent with a target marker can be observed by the propagation of acoustic shear waves in the substrate of the sensor. Phase and velocity of the acoustic wave are influenced by the specific adsorption of target markers onto the sensor surface. Piezoelectric materials, such as quartz (SiO₂) or zinc oxide (ZnO), resonate mechanically at a specific ultrasonic frequency when excited in an oscillating field. Electromagnetic energy is converted into acoustic energy, whereby piezoelectricity is associated with the electrical polarization of materials with anisotropic crystal structure. Generally, the oscillation method is used to monitor acoustic wave operation. Specifically, the oscillation method measures the series resonant frequency of the resonating sensor. Types of sensors derived from microgravimetric sensors include quartz crystal microbalance (QCM) devices that apply a thickness-shear mode (TSM) and devices that apply surface acoustic wave (SAW) detection principle. Additional devices derived from microgravimetric sensors include the flexural plate wave (FPW), the shear horizontal acoustic plate (SH-APM), the surface transverse wave (STW) and the thin-rod acoustic wave (TRAW).

### Conducting Polymers

Conducting polymer sensors promise fast response time, low cost, and good sensitivity and selectivity. The technology is relatively simple in concept. A conductive material, such as carbon, is homogeneously blended in a specific non-conducting polymer and deposited as a thin film on an aluminum oxide substrate. The films lie across two electrical leads, creating a chemoresistor. As the polymer is subjected to various chemical vapors, it expands, increasing the distance between carbon particles, and thereby increasing the resistance. The polymer matrix swells because analyte vapor absorbs into the film to an extent determined by the partition coefficient of the analyte. The partition coefficient defines the equilibrium distribution of an analyte between the vapor phase and the condensed phase at a specified temperature. Each individual detector element requires a minimum absorbed amount of analyte to cause a response noticeable above the baseline noise. Selectivity to different vapors is accomplished by changing the chemical composition of the polymer. This allows each sensor to be tailored to specific chemical vapors. Therefore, for most applications an array of orthogonal responding sensors is required to improve selectivity. Regardless of the number of sensors in the array, the information from them must be processed with pattern recognition software to correctly identify the chemical vapors of interest. Sensitivity concentrations are reportedly good (tens of ppm). The technology is very portable (small and low power consumption), relatively fast in response time (less than 1 minute), low cost, and should be rugged and reliable.

### Electrochemical Sensors

Electrochemical sensors measure a change in output voltage of a sensing element caused by chemical interaction of a target marker on the sensing element. Certain electrochemical sensors are based on a transducer principle. For example, certain electrochemical sensors use ion-selective electrodes that include ion-selective membranes, which generate a charge separation between the sample and the sensor surface. Other electrochemical sensors use an electrode by itself as the surface as the complexation agent, where a change in the electrode potential relates to the concentration of the target marker. Further examples of electrochemical sensors are based on semiconductor technology for monitoring charges at the surface of an electrode that has been built up on a metal gate between the so-called source and drain electrodes. The surface potential varies with the target marker concentration.

Additional electrochemical sensor devices include amperometric, conductometric, and capacitive immunosensors. Amperometric immunosensors are designed to measure a current flow generated by an electrochemical reaction at a constant voltage. Generally, electrochemically active labels directly, or as products of an enzymatic reaction, are needed for an electrochemical reaction of a target marker at a sensing electrode. Any number of commonly available electrodes can be used in amperometric immunosensors, including oxygen and H₂O₂ electrodes.

Capacitive immunosensors are sensor-based transducers that measure the alteration of the electrical conductivity in a solution at a constant voltage, where alterations in conductivity are caused by biochemical enzymatic reactions, which specifically generate or consume ions. Capacitance changes are measured using an electrochemical system, in which a bioactive element is immobilized onto a pair of metal electrodes, such as gold or platinum electrodes.

Conductometric immunosensors are also sensor-based transducers that measure alteration of surface conductivity. As with capacitive immunosensors, bioactive elements are immobilized on the surface of electrodes. When the bioactive element interacts with a target marker, it causes a decrease in the conductivity between the electrodes.

Electrochemical sensors are excellent for detecting low parts-per-million concentrations. They are also rugged, draw little power, linear and do not require significant support electronics or vapor handling (pumps, valves, etc.) They are moderate in cost ($50 to $200 in low volumes) and small in size.

### Gas Chromatography/Mass Spectrometry (GC/MS)

Gas Chromatography/Mass Spectrometry (GC/MS) is actually a combination of two technologies. One technology separates the chemical components (GC) while the other one detects them (MS). Technically, gas chromatography is the physical separation of two or more compounds based on their differential distribution between two phases, the mobile phase and stationary phase. The mobile phase is a carrier gas that moves a vaporized sample through a column coated with a stationary phase where separation takes place. When a separated sample component elutes from the column, a detector converts the column eluent to an electrical signal that is measured and recorded. The signal is recorded as a peak in the chromatogram plot. Chromatograph peaks can be identified from their corresponding retention times. The retention time is measured from the time of sample injection to the time of the peak maximum, and is unaffected by the presence of other sample components. Retention times can range from seconds to hours, depending on the column selected and the component. The height of the peak relates to the concentration of a component in the sample mixture.

After separation, the chemical components need to be detected. Mass spectrometry is one such detection method, which bombards the separated sample component molecules with an electron beam as they elute from the column. This causes the molecules to lose an electron and form ions with a positive charge. Some of the bonds holding the molecule together are broken in the process, and the resulting fragments may rearrange or break up further to form more stable fragments. A given compound will ionize, fragment, and rearrange reproducibly under a given set of conditions. This makes identification of the molecules possible. A mass spectrum is a plot showing the mass/charge ratio versus abundance data for ions from the sample molecule and its fragments. This ratio is normally equal to the mass for that fragment. The largest peak in the spectrum is the base peak. The GC/MS is accurate, selective and sensitive. Recent advances have reduced the size and cost of these devices to the point where small table top devices for use in healthcare facilities are now a reality. Further miniaturization and lower costs are likely to be achieved in the near future, as these devices are frequently used to detect weapons of mass destruction and need to be deployed in the field.

### Infrared Spectroscopy (FTIR, NDIR)

Infrared (IR) spectroscopy is one of the most common spectroscopic techniques used by organic and inorganic chemists. Simply, it is the absorption measurement of different IR frequencies by a sample positioned in the path of an IR beam. IR radiation spans a wide section of the electromagnetic spectrum having wavelengths from 0.78 to 1000 micrometers (microns). Generally, IR absorption is represented by its wave number, which is the inverse of its wavelength times 10,000. For a given sample to be detected using IR spectroscopy, the sample molecule must be active in the IR region, meaning that the molecule must vibrate when exposed to IR radiation. Several reference books are available which contain this data, including the Handbook of Chemistry and Physics from the CRC Press.

There are two general classes of IR spectrometers—dispersive and non-dispersive. In a typical dispersive IR spectrometer, radiation from a broadband source passes through the sample and is dispersed by a monochromator into component frequencies. The beams then fall on a detector, typically a thermal or photon detector, which generates an electrical signal for analysis. Fourier Transform IR spectrometers (FTIR) have replaced the dispersive IR spectrometer due to their superior speed and sensitivity. FTIR eliminates the physical separation of optical component frequencies by using a moving mirror Michelson interferometer and taking the Fourier transform of the signal.

Conversely, in the non-dispersive IR (NDIR) spectrometer, instead of sourcing a broad IR spectrum for analyzing a range of sample gases, the NDIR sources a specific wavelength which corresponds to the absorption wavelength of the target sample. This is accomplished by utilizing a relatively broad IR source and using spectral filters to restrict the emission to the wavelength of interest. For example, NDIR is frequently used to measure carbon monoxide (CO), which absorbs IR energy at a wavelength of 4.67 microns. By carefully tuning the IR source and detector during design, a high volume production CO sensor is manufactured. This is particularly impressive, as carbon dioxide is a common interferent and has an IR absorption wavelength of 4.26 microns, which is very close to that of CO.

NDIR sensors promise low cost (less than $200), no recurring costs, good sensitivity and selectivity, no calibration and high reliability. They are small, draw little power and respond quickly (less than 1 minute). Warm up time is nominal (less than 5 minutes). Unfortunately, they only detect one target gas. To detect more gases additional spectral filters and detectors are required, as well as additional optics to direct the broadband IR source. As with GC-MS, recent advances have reduced the size and cost of these devices to the point where small table top devices for use in healthcare facilities are now a reality. Further miniaturization and lower costs are likely to be achieved in the near future, as these devices are frequently used to detect weapons of mass destruction and need to be deployed in the field.

### Ion Mobility Spectrometry (IMS)

Ion Mobility Spectrometry (IMS) separates ionized molecular samples on the basis of their transition times when subjected to an electric field in a tube. As the sample is drawn into the instrument, it is ionized by a weak radioactive source. The ionized molecules drift through the cell under the influence of an electric field. An electronic shutter grid allows periodic introduction of the ions into the drift tube where they separate based on charge, mass, and shape. Smaller ions move faster than larger ions through the drift tube and arrive at the detector sooner. The amplified current from the detector is measured as a function of time and a spectrum is generated. A microprocessor evaluates the spectrum for the target compound, and determines the concentration based on the peak height.

IMS is an extremely fast method and allows near real time analysis. It is also very sensitive, and should be able to measure all the analytes of interest. IMS is moderate in cost (several thousand dollars) and larger in size and power consumption.

### Metal Oxide Semiconductor (MOS) Sensors

Metal Oxide Semiconductor (MOS) sensors utilize a semiconducting metal-oxide crystal, typically tin-oxide, as the sensing material. The metal-oxide crystal is heated to approximately 400° C., at which point the surface adsorbs oxygen. Donor electrons in the crystal transfer to the adsorbed oxygen, leaving a positive charge in the space charge region. Thus, a surface potential is formed, which increases the sensor's resistance. Exposing the sensor to deoxidizing, or reducing, gases removes the surface potential, which lowers the resistance. The end result is a sensor which changes its electrical resistance with exposure to deoxidizing gases. The change in resistance is approximately logarithmic.

MOS sensors have the advantage of being extremely low cost (less than $8 in low volume) with a fast analysis time (milliseconds to seconds). They have long operating lifetimes (greater than five years) with no reported shelf life issues.

### Thickness-Shear Mode Sensors (TSM)

TSM sensors consist of an AT-cut piezoelectric crystal disc, most commonly of quartz because of its chemical stability in biological fluids and resistance to extreme temperatures, and two electrodes (preferably metal) attached to opposite sides of the disc. The electrodes apply the oscillating electric field. Generally, TSM sensor devices are run in a range of 5-20 MHz. Advantages are, besides the chemical inertness, the low cost of the devices and the reliable quality of the mass-produced quartz discs.

### Photo-Ionization Detectors (PID)

Photo-Ionization Detectors rely on the fact that all elements and chemicals can be ionized. The energy required to displace an electron and ‘ionize’ a gas is called its Ionization Potential (IP), measured in electron volts (eV). A PID uses an ultraviolet (UV) light source to ionize the gas. The energy of the UV light source must be at least as great as the IP of the sample gas. For example, benzene has an IP of 9.24 eV, while carbon monoxide has an IP of 14.01 eV. For the PID to detect the benzene, the UV lamp must have at least 9.24 eV of energy. If the lamp has an energy of 15 eV, both the benzene and the carbon monoxide would be ionized. Once ionized, the detector measures the charge and converts the signal information into a displayed concentration. Unfortunately, the display does not differentiate between the two gases, and simply reads the total concentration of both summed together.

Three UV lamp energies are commonly available: 9.8, 10.6 and 11.7 eV. Some selectivity can be achieved by selecting the lowest energy lamp while still having enough energy to ionize the gases of interest. The largest group of compounds measured by a PID are the organics (compounds containing carbon), and they can typically be measured to parts per million (ppm) concentrations. PIDs do not measure any gases with an IP greater than 11.7 eV, such as nitrogen, oxygen, carbon dioxide and water vapor. The CRC Press Handbook of Chemistry and Physics includes a table listing the IPs for various gases.

PIDs are sensitive (low ppm), low cost, fast responding, portable detectors. They also consume little power.

### Surface Acoustic Wave Sensors (SAW)

Surface Acoustic Wave (SAW) sensors are constructed with interdigitated metal electrodes fabricated on piezoelectric substrates both to generate and to detect surface acoustic waves. Surface acoustic waves are waves that have their maximum amplitude at the surface and whose energy is nearly all contained within 15 to 20 wavelengths of the surface. Because the amplitude is a maximum at the surface such devices are very surface sensitive. Normally, SAW devices are used as electronic bandpass filters in cell phones. They are hermetically packaged to insure that their performance will not change due to a substance contacting the surface of the SAW.

SAW chemical sensors take advantage of this surface sensitivity to function as sensors. To increase specificity for specific compounds, SAW devices are frequently coated with a thin polymer film that will affect the frequency and insertion loss of the device in a predictable and reproducible manner. Each sensor in a sensor array is coated with a different polymer and the number and type of polymer coating are selected based on the chemical to be detected. If the device with the polymer coating is then subjected to chemical vapors that absorb into the polymer material, then the frequency and insertion loss of the device will further change. It is this final change that allows the device to function as a chemical sensor.

If several SAW devices are each coated with a different polymer material, the response to a given chemical vapor will vary from device to device. The polymer films are normally chosen so that each will have a different chemical affinity for a variety of organic chemical classes, that is, hydrocarbon, alcohol, ketone, oxygenated, chlorinated, and nitrogenated. If the polymer films are properly chosen, each chemical vapor of interest will have a unique overall effect on the set of devices. SAW chemical sensors are useful in the range of organic compounds from hexane on the light, volatility extreme to semi-volatile compounds on the heavy, low volatility extreme.

Motors, pumps and valves are used to bring the sample into and through the array. The sensitivity of the system can be enhanced for low vapor concentrations by having the option of using a chemical preconcentrator before the array. In operation, the preconcentrator absorbs the test vapors for a period of time and is then heated to release the vapors over a much shorter time span thereby increasing the effective concentration of the vapor at the array. The system uses some type of drive and detection electronics for the array. An on board microprocessor is used to control the sequences of the system and provide the computational power to interpret and analyze data from the array.

SAW sensors are reasonably priced (less than $200) and have good sensitivity (tens of ppm) with very good selectivity. They are portable, robust and consume nominal power. They warm up in less than two minutes and require less than one minute for most analysis. They are typically not used in high accuracy quantitative applications, and thus require no calibration. SAW sensors do not drift over time, have a long operating life (greater than five years) and have no known shelf life issues. They are sensitive to moisture, but this is addressed with the use of a thermally desorbed concentrator and processing algorithms.

### Amplifying Fluorescent Polymer Technology

Sensors can use fluorescent polymers that react with volatile chemicals as sensitive target marker detectors. Conventional fluorescence detection normally measures an increase or decrease in fluorescence intensity or an emission wavelength shift that occurs when a single molecule of the target marker interacts with an isolated chromophore, where the chromophore that interacts with the target marker is quenched; the remaining chromophores continue to fluoresce.

A variation of this approach is the “molecular wire” configuration, as described by Yang and Swager, J. Am. Chem. Soc., 120:5321-5322 (1998) and Cumming et al., IEEE Trans Geoscience and Remote Sensing, 39:1119-1128 (2001), both of which are incorporated herein by reference in their entirety. In the molecular wire configuration, the absorption of a single photon of light by any chromophore will result in a chain reaction, quenching the fluorescence of many chromophores and amplifying the sensory response by several orders of magnitude. Sensors based on the molecular wire configuration have been assembled for detecting explosives (see Swager and Wosnick, MRS Bull, 27:446-450 (2002), which is incorporated herein by reference in its entirety.

### Fiber Optic Microsphere Technology

Fiber optic microsphere technology is based upon an array of a plurality of microsphere sensors (beads), wherein each microsphere belongs to a discrete class that is associated with a target marker, that is placed on an optical substrate containing a plurality of micrometer-scale wells (see, for example, Michael et al., Anal Chem, 71:2192-2198 (1998); Dickinson et al., Anal Chem., 71:2192-2198 (1999); Albert and Walt, Anal Chem, 72:1947-1955 (2000); and Stitzel et al., Anal Chem, 73:5266-5271 (1001), all of which are incorporated herein by reference in their entirety). Each type of bead is encoded with a unique signature to identify the bead as well as its location. Upon exposure to a target marker, the beads respond to the target marker and their intensity and wavelength shifts are used to generate fluorescence response patterns, which are, in turn, compared to known patterns to identify the target marker.

### Interdigitated Microelectrode Arrays (IME)

Interdigitated microelectrode arrays are based on the used of a transducer film that incorporates an ensemble of nanometer-sized metal particles, each coated by an organic monomolecular layer shell (see, for example, Wohltjen and Snow, Anal Chem, 70:2856-2859 (1998); and Jarvis et al., Proceedings of the 3^{rd} Intl Aviation Security Tech Symposium, Atlantic City, N.J., 639-647 (2001), both of which are incorporated herein by reference in their entirety). Such sensor devices are also known as metal-insulator-metal ensembles (MIME) because of the combination of a large group of colloidal-sized, conducting metal cores separated by thin insulating layers.

### Microelectromechanical Systems (MEMS)

Sensor technology based on MEMS integrate mechanical elements, sensors, actuators, and electronics on a common silicon substrate for use in detecting target markers (see, for example, Pinnaduwage et al., Proceedings of 3^{rd} Intl Aviation Security Tech Symposium, Atlantic City, N.J., 602-615 (2001); and Lareau et al., Proceedings of 3^{rd} Intl Aviation Security Tech Symposium, Atlantic City, N.J., 332-339 (2001), both of which are incorporated herein by reference in their entirety).

One example of sensor technology based on MEMS is microcantilever sensors. Microcantilever sensors are hairlike, silicon-based devices that are at least 1,000 times more sensitive and smaller than currently used sensors. The working principle for most microcantilever sensors is based on a measurement of displacement. Specifically, in biosensor applications, the displacement of a cantilever-probe is related to the binding of molecules on the (activated) surface of the cantilever beam, and is used to compute the strength of these bonds, as well as the presence of specific reagents in the solution under consideration (Fritz, J. et al., “Translating biomolecular recognition into nanomechanics,” Science, 288:316-318 (2000); Raiteri, R. et al., “Sensing of biological substances based on the bending of microfabricated cantilevers,” Sensors and Actuators B, 61:213-217 (1999), both of which are incorporated herein by reference in their entirety). It is clear that the sensitivity of these devices strongly depends on the smallest detectable motion, which poses a constraint on the practically vs. theoretically achievable performance.

One example of microcantilever technology uses silicon cantilever beams (preferably a few hundred micrometers long and 1 μm thick) that are coated with a different sensor/detector layer (such as antibodies or aptamers). When exposed to a target marker, the cantilever surface absorbs the target marker, which leads to interfacial stress between the sensor and the absorbing layer that bends the cantilever. Each cantilever bends in a characteristic way typical for each target marker. From the magnitude of the cantilever's bending response as a function of time, a fingerprint pattern for each target marker can be obtained.

Microcantilever sensors are highly advantageous in that they can detect and measure relative humidity, temperature, pressure, flow, viscosity, sound, ultraviolet and infrared radiation, chemicals, and biomolecules such as DNA, proteins, and enzymes. Microcantilever sensors are rugged, reusable, and extremely sensitive, yet they cost little and consume little power. Another advantage in using the sensors is that they work in air, vacuum, or under liquid environments.

### Molecularly Imprinted Polymeric Film

Molecular imprinting is a process of template-induced formation of specific molecular recognition sites (binding or catalytic) in a polymeric material where the template directs the positioning and orientation of the polymeric material's structural components by a self-assembling mechanism (see, for example, Olivier et al., Anal Bioanal Chem, 382:947-956 (2005); and Ersoz et al., Biosensors & Bioelectronics, 20:2197-2202 (2005), both of which are incorporated herein by reference in their entirety). The polymeric material can include organic polymers as well as inorganic silica gels. Molecularly imprinted polymers (MIPs) can be used in a variety of sensor platforms including, but not limited to, fluorescence spectroscopy; UV/Vis spectroscopy; infrared spectroscopy; surface plasmon resonance; chemiluminescent adsorbent assay; and reflectometric interference spectroscopy. Such approaches allow for the realization of highly efficient and sensitive target marker recognition.

### EXAMPLE 4—DETECTION OF GLUCOSE IN EXHALED BREATH

Persons with diabetes presently check their blood glucose levels between 1 and 6-8 times each day. Knowledge of blood glucose levels is an absolute necessity for guiding proper administration and dosing of insulin and other drugs used to control hyperglycemia. Presently the person must draw blood samples, usually from a finger using a lancet device, and place the sample on a “test strip” which is inserted into a glucose monitor that gives the blood glucose concentration. This process requires considerable skill, time and subjects the person with diabetes to immediate recognition as a diabetic and thus results in the potential for embarrassment and even prejudice and/or discrimination when applying for employment.

An attractive alternative is to use a sensor system that collects a sample of exhaled breath which for compounds such as glucose, which are extremely hydrophilic, condenses the sample into a “condensate” which is then placed in contact with the sensor by a pump or microfluidic system. Thus, persons with diabetes are far more likely to inconspicuously blow into a small hand-held device that provides a blood glucose concentration from an exhaled breath sample then to perform the multiple steps required for a blood sample, particularly in public places. This technology is likely to increase the acceptance of frequent blood glucose monitoring and reduce the embarrassment that many persons with diabetes feel when having to draw blood samples from their fingers. Adherence to frequent blood glucose testing and subsequent administration of appropriate doses of insulin have been shown to dramatically reduce the incidence of complications related to diabetes. Thus, frequent monitoring of exhaled breath glucose is a means to show adherence to a strict regimen to reduce the incidence of complications related to poor diabetes control.

It should be understood that the examples and embodiments described herein are for illustrative purposes only and that various modifications or changes in light thereof will be suggested to persons skilled in the art and are to be included within the spirit and purview of this application and the scope of the appended claims. Specifically, the marker detection method of the present invention is intended to cover detection not only through the exhalation by a subject with a device utilizing electronic nose technology, but also other suitable technologies, such as gas chromatography, transcutaneous/transdermal detection, semiconductive gas sensors, mass spectrometers, IR or UV or visible or fluorescence spectrophotometers.

All patents, patent applications, provisional applications, and publications referred to or cited herein, or from which a claim for benefit of priority has been made, are incorporated by reference in their entirety to the extent thy are not inconsistent with the explicit teachings of this specification.

We claim:

1. A system for monitoring subject drug adherence for at least one prescribed drug regimen comprising:
a) a means for obtaining a sample of a subject's breath at prescribed intervals;
b) a sensor for detecting at least one marker in the breath sample indicative of a subject taking the drug wherein the sensor generates an output signal indicative of the presence or absence of the marker in the breath sample;
c) a processing means for receiving the output signal and assessing the concentration of marker present in the breath sample for use in ascertaining subject drug adherence with the drug regimen(s) as prescribed.

2. The system of claim 1, wherein the processing means conducts a comparison of the marker concentration against a predetermined marker concentration of the drug marker to determine drug adherence to the prescribed drug regimen(s).

3. The system of claim 1, further comprising a means for reporting results obtained from the processing means.

4. The system of claim 1, further comprising a means for dispensing the prescribed drug.

5. The system of claim 1, wherein multiple drug regimens are prescribed to the subject.

6. The system of claim 1, wherein the sensor is selected from the group consisting of: High Electron Mobility Transistors (HEMT), metal-insulator-metal ensemple (MIME) sensors, nuclear magnetic resonance (NMR), cross-reactive optical microsensor arrays, fluorescent polymer films, surface enhanced Raman spectroscopy (SERS), diode lasers, selected ion flow tubes, metal oxide sensors (MOS), bulk acoustic wave (BAW) sensors, colorimetric tubes, infrared spectroscopy, gas chromatography-mass spectroscopy (including “miniature” gas chromatography), semiconductive gas sensor technology; mass spectrometers, fluorescent spectrophotometers, conductive polymer gas sensor technology; aptamer sensor technology; amplifying fluorescent polymer (AFP) sensor technology; surface acoustic wave gas sensor technology; or quantum cascade lasers.

7. The system of claim 1, wherein the system is portable.

8. The system of claim 1, wherein the drug marker is detectable in breath upon enzymatic degradation of the drug marker in the subject.

9. The system of claim 1, wherein the breath drug marker is detectable in breath upon spontaneous non-enzymatic conversion of the drug marker in the subject.

10. The system of claim 1, wherein the breath drug marker is detectable in breath without further chemical modification of the drug marker.

11. The system of claim 1, wherein the breath drug marker is the drug, a metabolite of the drug, an additive delivered with the drug regimen, or a metabolite of an additive delivered with the drug.

12. A method for monitoring subject drug adherence with at least one prescribed drug regimen comprising:
a) a means for obtaining a sample of a subject's breath at prescribed intervals;
b) analyzing the sample with sensor technology to identify the presence of at least one drug marker in the sample; and
c) determining the presence of at least one drug marker at a level above a predetermined threshold, which indicates subject adherence to the prescribed drug regimen.

13. The method of claim 12, further comprising the step of d) based on the results generated from step (c), reporting whether subject has adhered to a prescribed drug regimen.

14. The method of claim 12, further comprising the step of altering, maintaining, or canceling the prescribed drug regimen based on the results generated from step (c).

15. The method of claim 12, further comprising the step of assessing the subject's health status by evaluating their pattern of adherence to the prescribed drug regimen.

16. The method of claim 12, further comprising any one or combination of the following steps: identifying a subject with a condition treatable with a prescribed drug regimen; prescribing a drug regimen for the subject; defining an acceptable level of adherence with the prescribed drug regimen; and assessing indicators of progression of patient condition while taking the prescribed drug regimen.

17. The method of claim 12, wherein the step of obtaining the breath sample is performed at each and every prescribed administration of the drug.

18. The method of claim 12, wherein the step of obtaining the breath sample is performed at a prescribed frequency other than every prescribed administration of the drug.

19. The method of claim 12, further comprising the step of obtaining a breath sample prior to administration of the drug to establish a baseline for comparison.

20. The method of claim 12, further comprising the step of administering a drug at prescribed intervals to the subject, wherein the drug is administered orally, via inhalation, intraocularly, transdermally, intravenously, intraperitoneally, or vaginally.

21. A kit for monitoring subject adherence with at least one prescribed drug regimen comprising:
a) a means for dispensing a prescribed drug;
b) a housing;
c) a sensor disposed within the housing, said sensor having the ability to detect the presence of at least one drug marker in breath; and
d) a reporting module disposed with or within the housing, wherein said reporting module is operatively connected to the sensor such that detection of the presence of the drug marker in the sample is communicated at prescribed intervals.

22. The kit of claim 21, wherein the drug comprises a marker that is detectable in breath upon enzymatic breakdown of the drug.

23. The kit of claim 21, wherein the drug comprises a marker that is detectable in breath upon spontaneous non-enzymatic activity on the drug in the subject

24. The kit of claim 21, wherein the breath drug marker is the drug, a metabolite of the drug, an additive delivered with the drug regimen, or a metabolite of an additive delivered with the drug.

25. The kit of claim 21, wherein the drug marker is selected from the group consisting of: dimethyl sulfoxide (DMSO), acetaldehyde, acetophenone, trans-Anethole (1-methoxy-4-propenyl benzene) (anise), benzaldehyde (benzoic aldehyde), benzyl alcohol, benzyl cinnamate, cadinene, camphene, camphor, cinnamaldehyde (3-phenylpropenal), garlic, citronellal, cresol, cyclohexane, eucalyptol, and eugenol, eugenyl methyl ether; butyl isobutyrate (n-butyl 2, methyl propanoate) (pineapple); citral (2-trans-3,7-dimethyl-2,6-actadiene-1-al); menthol (1-methyl-4-isopropylcyclohexane-3-ol); α-Pinene (2,6,6-trimethylbicyclo-(3,1,1)-2-heptene); flouroalcohols; and fluoroaldehydes.
